(19) United States
(12) Patent Application Publication
(10) Pub. No.: US 2024/0122811 A1
(43) Pub. Date: Apr. 18, 2024

(54) DEVICE FOR FLOW DETECTION OF MOTHER'S MILK

(71) Applicant: KAIZEN BIO-TECH (2011) LTD., Caesarea (IL)

(72) Inventors: Haim DAHAN, Raanana (IL); Adir KAN, Natanya (IL); Oded HADOMI, Even Yehuda (IL); Abi ZAKAY, Tel Aviv (IL); Mario MESCHIANY, Tel Aviv (IL)

(21) Appl. No.: 18/277,383

(22) PCT Filed: Feb. 16, 2022
(86) PCT No.: PCT/IB2022/051373

§ 371 Date: Aug. 16, 2023

Related U.S. Application Data
(60) Provisional application No. 63/223,616, filed on Jul. 20, 2021.

(30) Foreign Application Priority Data
Feb. 16, 2021 (IL) 280929

(51) Int. Cl. A61J 13/00; A61J 7/00

(52) U.S. Cl. CPC A61J 13/00; A61J 7/0053; A61J 2200/70; A61J 2205/20; A61J 2205/70

(57) ABSTRACT

New devices for providing an indication to a nursing mother as to whether a baby is receiving milk during a breastfeeding session. Milk expressed from the mother's breast is caused to flow through a transparent passageway part of a nipple shield, disposed in in a position where it visible to the mother or a third party, before being supplied to a nipple of the device, from which the baby is enabled to suck. The device therefore provides an indication that the infant is receiving milk from the mother's breast. The visibility of the passageway is achieved by routing the passageway through a region of the device whose line of sight with the mother is not obscured by the baby when feeding, such as towards the periphery of the base layer of the device. The device passageway may also comprise a valve, and details of such valves are disclosed.

### FIELD

The present disclosure describes technology related to the field of the detection of the flow of milk from a nursing mother, especially for indicating the presence of that flow to the mother.

### BACKGROUND

The desire to receive an indication of whether a baby is actually intaking milk during a breastfeeding session often arises, especially in the first few weeks of an infant's life. Babies may be inclined to suck on a breast as a soothing mechanism, giving the mother the impression that the baby is intaking milk, when in actual fact, they are not. Additionally, some mothers do not have an adequate milk supply, especially before breast-feeding has been well-established, and therefore a hungry infant may suckle on the breast for long periods of time, without receiving an adequate supply of milk. Additionally, some babies appear to fall asleep during nursing, and the mother may not realize that they are still feeding.

In order to give an indication of whether milk is being supplied to a baby, various complex measurement systems have been proposed. One technique employed is weight subtraction, which is bothersome, time consuming and inaccurate. In this technique, the baby's weight is measured before and after breastfeeding, and the amount of milk consumption is calculated by subtracting the two weights. Another technique is disclosed in the article by S. E. J. Daly et al., entitled “The Determination of Short-Term Breast Volume Changes and the Rate of Synthesis of Human Milk Using Computerized Breast Measurement” published in Exp. Physiology, 77, 79-87 (1992). In this technique, changes in breast volume are traced by computerized imaging of the breast before and after feeding. International Patent Publication No. WO 2006/054287 for “Breast Milk Flow Meter Apparatus and Method” by E. Kolberg et al, disclose a technique in which a volumetric flow sensor is placed inside a silicon nipple cap through which the baby suckles. The milk flow data from the sensor is converted into milk volume data which is displayed to the mother. Such systems generally involve attaching electronic or electromechanical flow meters to a fluid flow passage in order to measure the fluid flow. There are several other systems proposed which use electronic flow measurement modules, attached externally to a milk collection device which fits over the mother's breast, to measure the milk flow. One such system is shown in US. Published Application No. 2018/0147124 for “Non-Intrusive Breast Milk Monitoring” to L. A. Drew, which has an electronic sensor to measure the milk flow. However, all of the above mentioned systems are expensive and complex, because of the electronic milk sensing unit, making their suitability for a low-cost, possibly disposable device unlikely. Also, many mothers would be hesitant to use electronic devices on or near the baby, generally because of their fear of the presence of any electromagnetic radiation, and there is the additional danger that such externally attached parts may become disconnected from the device and somehow ingested by the infant. A simpler device resembling a nipple shield, has been described in U.S. Pat. No. 7,896,835 for “Apparatus and Method for Measuring Fluid Flow to a Suckling Baby”, commonly owned by the present applicant, in which a fraction of the main milk flow is passed through a measurement channel, where the length of the milk collected in the measurement channel after the feeding session, provides a measure of the milk drawn through the main channel. As the benefits of breast-feeding have become widely known, more mothers are breast-feeding than in the past, highlighting the need for a method to indicate a baby's milk intake.

The present disclosure attempts to provide a novel device and method that overcomes at least some of the disadvantages of prior art systems and methods.

The disclosures of each of the publications mentioned in this section and in other sections of the specification, are hereby incorporated by reference, each in its entirety.

### SUMMARY

The present disclosure describes new exemplary devices for providing an indication to a nursing mother as to whether a baby is receiving milk during a breastfeeding session. The device is constructed of a flexible layer, which typically has a shape and design similar to that of nipple shields currently available, and the layout of the flow geometry of the device is such as to cause milk expressed from the mother's breast to flow through a passageway having a region which provides an indication of milk flow, before being supplied to the nipple of the device from which the baby is sucking. The indicator part of the passage should be located in a position where it is accessible to the mother or to an assistant. A simple way of implementing the indicator part of the passageway is by having that part constructed of a transparent or translucent material, such that the milk flow is visible to the mother, and this implementation will be used hereinbelow when describing the device, but without intending to limit the flow indication to a visible indication. The passageway may be disposed within the device, or at the surface of the device, or attached otherwise to the device. The device therefore provides the mother, or a third party, with an indication that the infant is receiving milk from the mother's breast. An alternative method of constructing the device, eliminates the need for the milk to pass through a passageway, by providing a small indicating container connected fluidly to the space for the milk flow between the mother's breast and the nipple of the device, such that a portion of the milk flowing directly to the baby, passes along the connection to the indicating container, where the presence or not of milk, is visible to the mother. The milk flow could also be shown by the use of a mechanical protrusion into the flow channel of the milk, the flow causing the mechanical protrusion to change its orientation, thereby indicating the milk flow. In yet another implementation, a small closed container can be attached by means of a short connection tube to the milk passageway at a position remote from the baby and the mother's nipple, and when the milk flows in the passageway as the baby sucks, milk surges along the connection tube and into the closed container, where it can be readily seen.

The device is advantageously constructed of a flexible layer, which typically has a shape and design similar to nipple shields currently available. The flexible layer is typically constructed from a material which is non-absorbent and not irritating to the skin, such as silicone. Like a nipple shield, the flexible layer is adapted to be fitted conformally over a part of a woman's breast, covering the areola and nipple, with a central protrusion, hereinbelow also referred to as the device nipple, adapted to be positioned over the mother's nipple. This protrusion is so named because of its geometrical form when viewed from the outside, though it also has the functional form of a concave region or recess when viewed from the side of the mother's breast. This feature may be alternately called by either name, depending on the context in which it is being referred to, generally whether relating to the functionality from the inside of the device, or that from the outside of the device, as will be apparent hereinbelow. However, this convention is not to be understood as limiting such a functional use or relationship, and both terms or another descriptive name may be used alternatively. The protrusion/concave region may be constructed as an integral part of the flexible layer, or it may be attached in the central region of the flexible layer, and even be constructed of another material. Different shaped and different sized devices may be provided to fit the individual user, such that during application of the device, the flexible layer fits conformally over the user's breast.

In order to provide an indication of whether the baby is receiving milk during a feeding session, the device may have one or more passageways, which can have a round cross section, or any other suitable shape, for visually displaying the flow of milk, and especially, the surges of milk mimicking the sucking pattern of the baby. The passageway is typically embedded within the flexible layer, such that the device can be formed of one piece, or it can be formed on the surface of the flexible layer, or affixed above the surface and attached at its ends. The passageway has two openings, the first opening from the central concave region of the device, such that when the device is worn, the opening is facing the mother's breast. The second opening or openings of the passageway is also located within the central concave region of the device, but in the apex region of the protrusion, as an opening to the outer surface of the flexible layer such that it faces the baby during breastfeeding. This opening to the outside should not have fluid contact with the hollow internal volume of the central concave hollow. Between the two openings, the passageway performs a circuitous path radially outward from the central concave recess region, such that when the device is worn by the mother, a part of the passageway is located in a region which should be visually unobstructed by the mouth of the baby, and thus visible to the mother or a third party during use of the device. The flexible layer should be formed of a transparent or translucent material, such that fluid flow through the passageway is visible to a user. If not, at least a part of the flexible layer in which the passageway is embedded should be constructed from a transparent or translucent material, such that part of the passageway is visible during breastfeeding. Generally, the second opening, which operates as an exit aperture is disposed at the apex of the protrusion, mimicking the mother's nipple structure. The passageway may diverge into a number of branches in the region of the second opening, so that the second “opening” may be composed of a number of separate apertures. This arrangement should enable the baby to suck more efficiently, since no part of the baby's tongue or lips can then occlude the entire opening, as may be the case with a single opening. Likewise, the entry of the passageway within the concave recess may have multiple openings. Furthermore, the device may be provided with more than one parallel passageway to ensure that a sufficient flow of milk is enabled. This may be necessary, for instance, when the passageway is formed within the thin flexible layer of the device, and therefore has a limited cross section, such that the flow through a single passageway would be limited. At least one of the parallel passageways requires a region at which the mother can obtain a visible indication of the milk flow. In order to simplify the description, any number of passageways will be referred to as a single passageway hereinbelow, and therefore should be understood as claimed, to include also a number of passageways, without intending to limit the application.

When the device is used, the flexible layer fits conformally to the woman's breast, with the concave region, which is the device nipple, positioned over the nipple of the mother. There is typically a hollow space between the mother's nipple and the device's nipple. During breastfeeding, the baby sucks on the outer side of the protrusion—the device nipple—thus creating negative pressure in the passageway. The negative pressure generates a vacuum in the passageway, assisting in drawing milk from the mother's nipple into the hollow concave space and the passageway, and drawing the milk from the hollow concave space and the passageway to provide the baby with the milk. The negative pressure forces the flexible layer to adhere to the mother's breast, thereby preventing milk from escaping externally from the concave space and from the outer rim of the device, and also reducing the entry of air through the outer rim and causing air bubbles in the milk flow.

Since the baby's mouth may occlude a large part of the flexible layer, the passageway must be disposed in a way that at least a part of it is visible even though the central region of the flexible layer may be visually blocked by the baby. The passageway is thus typically embedded in or mounted on the surface of, or otherwise attached to the flexible layer in such a position that it runs around at least a part of the rim region of the flexible layer, or at least in an area of the flexible layer outside the concave recess region, or through an outside location so that it is visible during a breastfeeding session.

The passageway may advantageously be provided with a valve opening at a predetermined pressure so that milk flow is enabled through the passageway in the direction from the opening in the central concave hollow towards the exit aperture in the apex of the device nipple to the baby. Several examples of such valves are provided in the Detailed Description section of this disclosure. Such a valve has the effect of assisting in the maintenance of the negative pressure within the passageway, and hence within the hollow concave region when the baby stops sucking, thus ensuring better adherence of the device to the mother's breast. In addition, the use of such a valve decreases the suction effort needed by the baby, since between pauses in the baby's sucking, the baby does not have to generate the entire sub-pressure needed to draw the milk from the concave space, which could be at atmospheric pressure without such a valve, to the baby's mouth, but only from the sub-pressure maintained in the concave space by the valve. This valve thus acts as a flow amplifier, and may reduce the time needed for the feeding session. Additionally, the negative pressure maintained within the concave milk collection space, may assist the mother by inducing a better flow of milk from the mother's breast. The valve can be fitted at any location along the length of the passageway, and models with various opening pressures can be provided to suit the sucking strength which the baby shows. A baby with more developed sucking abilities may advantageously use a valve opening at a higher absolute pressure. On the other hand, as an alternative consideration, the valve opening pressure can be selected in order to partly relieve the sub-atmospheric pressure that would be generated by the baby's sucking, so that it does not cause undue discomfort to the mother. The level selected can be chosen according to the mother's level of acceptance of the negative pressure applied to her nipples. If deemed necessary or advantageous, more than one valve may be used along the passageway to control flow. It should be clarified that references in this disclosure to opening or closing pressures, relate to the absolute pressure difference across the valve at which the valve opens or closes, independently as to whether the valve is situated in a negative pressure region, i.e. in a region of partial vacuum, or in a positive pressure region, i.e. in a region above atmospheric pressure.

According to a further exemplary implementation of these flow indicating devices, a two-way valve may also be used for this purpose, such a two-way valve being operative to avoid excess pressure applied to the mother's nipples from a baby's vigorous sucking, while at the same time, having the additional feature of maintaining a predetermined negative pressure within the device in order to keep the device attached to the mother's breast. The mechanism of this use is that when the baby stops sucking, if only a one-way valve were to be used, closing when the baby stops sucking, the high sub-pressure generate within the device would be maintained, thus continuing to exert the negative pressure on the mother's nipple. The use of a two-way valve ensures that when the baby stops sucking and the negative the generation of the high negative pressure by the baby within the device ceases, the reverse flow direction of the two-way valve allows air and/or milk to flow back into the passageway, but only up to the predefined pressure at which the valve is designed to close. Once the valve closes, the sub-pressure at this closing value is maintained but it is less than the sub-pressure generated by the baby, thereby relieving discomfort to the mother but not releasing all of the vacuum, such that the device remains seated on the mother's nipple.

All of the above described milk detection devices according to various implementations of the present disclosure have one common feature, which distinguishes them from previous devices such as those performing measurements on the milk flow, and that is that since the currently described devices only involve the addition of one or more simple milk passageways to a nipple shield structure, they can be produced at low cost by conventional polymer production techniques, without the need for any additional mechanical or electronic components. This feature is of great importance since it enables the devices to be constructed as unitary and compact, self-contained devices, of sufficiently low manufacturing cost as to become single use, disposable milk flow indication device. This is a significant advantage over previously described mechanical or electronic devices, and enables more universal use of milk indicating devices for the nursing mother. Furthermore, the construction as a unitary integral device avoids the danger that external parts may become detached with the fear that the nursing infant may ingest such a detached part. Additionally, an electronics-less device capable of providing information about milk flow to the baby, constructed only of inert polymer materials, should allay fears of a mother about operation of electronic devices so close to a young baby.

There is further described, according to another aspect of the present disclosure, a novel method of constructing such a two-way differential valve, self-actuated by the pressure difference across the valve, the opening pressure of which may be different for the two directions of flow. The valve uses a flexible diaphragm disposed across the direction of flow of the fluid, which has a different flexibility for the two directions of flow. This difference of flexibility is generated by providing a different bending length of the diaphragm, between the point of support of the diaphragm in the valve body, and its freely movable inner or outer periphery. The diaphragm is constrained between an inner post having an annular shoulder constraining the inner edge of the diaphragm, such that the diaphragm can only bend in one direction towards its outer edge, and an outer shoulder, constraining the outer edge of the diagram, such as the diaphragm can only bend in the other direction towards its inner edge. Adjustment of the length of the free diameter of the part of the diaphragm that can bend enables adjustment of the pressure across the valve which causes it to open. A fuller explanation of the operation of the valve is shown in the detailed description section in FIGS. 5A to 5E.

According to yet another implementation of this application, the described use of a valve controlling the flow of milk out of a nipple opening, enables the provision of a new nipple shield device, having the advantage that it can assist in ensuring that the nipple shield remains in place on the mother's breast when the baby stops sucking. The valve enables unobstructed flow of milk from the mother to the baby, but when the baby stops sucking, the valve closes and prevents the inflow of air back through the nipple openings of the nipple shield, which would release the vacuum holding the nipple shield in place, causing it to loose contact with the mother's breast. The valve may additionally and advantageously have an opening pressure difference which will nevertheless allow a certain amount of air to enter the nipple opening, to reduce the negative pressure on the mother's nipple, such that it is less uncomfortable to the mother, yet without releasing all of the vacuum level within the device which would cause it to become detached. For example, the breastfeeding baby may apply a negative suction pressure at a first level, but once the baby stops or pauses, the valve may allow air to enter to adjust the internal pressure on the mother's nipple to a less negative level, which is less uncomfortable to the mother. Additionally, the base of the nipple region of the device may be reinforced, such as by making the material thicker or stronger, such that the base of the nipple remains more firmly attached to the mother's breast, with a reduced tendency to bend and lift off, which would allow release of the vacuum.

A further inventive addition to any of the devices described in this application involves the use of a fluid dispenser incorporated into the milk flow detector, which could be advantageously used as a medicament dispenser. As is known, it is a challenge to give drugs to babies. The amount of medicine given should be accurate and the usual use of a spoon or a syringe to feed the baby the medicine is unreliable and inaccurate, since besides the problems of getting the baby to ingest the dose, he/she may also spit out part of the quantity. According to the present feature of this disclosure, a drug container reservoir is fitted to the device and fluidly connected to the milk passageway, such that once the container has been filled with the correct quantity of medication, the flow of the milk will be mixed with the drug in controlled way, depending on the flow rate of the medicine from the container reservoir to the milk passageway. The baby will then receive all of the drug dose, or any other fluid it is desired to provide the baby with, mixed with the mother's milk during breast feeding, such that even the taste of a drug would be masked. The container reservoir may be detachable for easy filing. Besides being applicable for incorporation into the milk flow detector of the present application, such a feature can also be incorporated into any other device which provides a flow passageway for the milk taken by the baby, such as the above referenced U.S. Pat. No. 7,896,835 for “Apparatus and Method for Measuring Fluid Flow to a Suckling Baby” having a common inventor with the present application. In addition, such a feature could also be incorporated into the improved nipple shield described hereinabove.

The milk flow detectors described hereinabove are all single function, unitary devices, for providing the mother with an indication that the baby is receiving a flow of her milk. Additionally, the added component parts have generally been described as part of such a single application device. According to further implementations of the devices described in the present application, a novel multi-task nipple shield is described, that can be used for performing a number of alternative functions related to different aspects of a nursing mother's needs. The nipple shield comprises a universal base unit which is fitted over the mother's breast, and which executes the transfer of the mother's milk from her nipple to the baby's mouth, by means of a passageway which conveys the milk to and from a location remote from the nipple. At this remote location, any of a number of different operational heads can be attached, each type of head being adapted to perform its own dedicated function related to the milk. The remote location includes a standardized pair of fluid flow connection terminals, and the various operational heads have matching standardized fluid flow connectors that may be attached to the remote connectors on the base nipple shield. The base unit of the nipple shield is thus universal, and the particular use made of the device depends on the head attached to the remote terminals of the nipple shield. Heads can be attached for various measurement or indicational functions, such as flow measurement, flow indication, medicine addition, measurement of the suction pattern of the baby, milk quality analysis, detection of markers in the milk indicating illness of the mother, and numerous other functional uses. Besides the multi-tasking use of the entire device concept, another advantage of such a device is that the base nipple shield, having channels and parts readily cleanable using a brush or a machine, can be made for multiple use, while the head attachments, being more complex and hence more difficult to clean, can be one-time use disposable units, though it is feasible that the head attachments can be made such that they can be cleaned and reused, especially those having electronic chips incorporated, and therefore somewhat costly to be considered a disposable attachment.

Although reference is made throughout this application and in the claims, to the mother of the baby as being the supplier of the milk, this being the usual situation, it is to be understood that references to the mother are not intended to exclude a woman other than the baby's mother, and the disclosure and the claims are not intended to be interpreted to be limited to a mother using the device to breast-feed her baby.

Although the devices described in this application are most usefully used for feeding from the mother's breast, they can also be produced in forms which can be bottle mounted, so that direct evidence of the milk flow is obtained while the baby is feeding, rather than interrupting the feeding session to hold the bottle upright to read the milk level on the graduations of the bottle. This embodiment also enables the use of opaque bottles.

There is thus provided in accordance with an exemplary implementation of the devices described in this disclosure, a device adapted to provide an indication of milk flow from a woman's breast to a baby, the device comprising:

a flexible layer adapted to fit over at least a portion of the woman's breast, the flexible layer comprising:
(i) an inner surface adapted to face the woman's breast, and an outer surface;
(ii) a protrusion disposed in a central region of the flexible layer, and adapted to be positioned over the nipple of the breast, and
(iii) at least one fluid communication passageway connecting the inner surface of the protrusion with at least one opening in the outer surface in the apex region of the protrusion, such that milk collected in a space between the nipple and the inner surface of the protrusion can flow through the at least one passageway and out of the at least one opening in the apex region, at least one portion of at least one passageway being disposed at a distance from the protrusion, such that during use of the device, the at least one portion is disposed outside of the area where the baby's lips are anticipated to be during use of the device.

In the above device, the at least one passageway may be either embedded within the flexible layer, or may be partially embedded in the flexible layer, or may be disposed on the outer surface of the flexible layer, or may be partially detached from the flexible layer.

According to further implementations, in any of the above described devices, at least one part of at least one passageway is either transparent or translucent, such that a flow of milk in the at least one part of at least one passageway is visible. Furthermore, the devices may comprise a closed at least partially transparent container connected to the at least one part of the at least one passageway, such that milk passing through the at least one passageway is visible surging in the at least partially transparent container. Additionally, the at least one part of the at least one passageway may comprise a mechanical element which undergoes deflection in a flow of milk. Even furthermore, the at least one part of the at least one passageway may have a property which indicates when a predetermined flow of milk exists within the at least one part. Such a property may be either a change of color or an emitted sound.

In any of the above described devices, the distance of the at least one portion of the at least one portion of the at least one passageway from the protrusion, may be at least 3 cm., such that the at least one portion is visible outside of the area where the mouth of a baby sucking on the protrusion is expected to be.

According to yet further implementations, any of these devices may further comprise at least one valve disposed along the at least one passageway and adapted to enable milk to flow through the passageway only from the space to the at least one opening in the apex region. In such a case, the at least one valve essentially closes when suction is not applied at the least one opening in the apex region.

Any of the above described devices may further comprise a container fluidly connected to at least one passageway, such that a fluid in the container can be provided to the baby. The fluid may be a medicament. Furthermore, the traversability of the fluid connection should be such that the contents of the fluid container are transferred to the milk flow in the at least one passageway according to a predetermined flow rate.

Any of the above described devices should be electronics-free. Consequently, the electronics-free status of the devices reduces the risk of electro-magnetic radiation in the region of the baby.

There is further provided according to a further implementation of the present disclosure, a device adapted to be fitted over at least the nipple region of a woman's breast, for providing an indication of milk flow from the breast, the device comprising:
(i) a flexible layer of material having a nipple shaped protrusion with an inner surface and an outer surface, the inner surface adapted to face the nipple region of the woman's breast, and the outer surface adapted to face the mouth of a baby; and
(ii) at least one passageway formed within the flexible layer, the at least one passageway passing from a first location on the inner surface of the nipple-shaped protrusion, to the outer surface of the flexible layer at or near the apex of the nipple shaped protrusion,
   wherein at least one portion of the at least one passageway is adapted to provide an indication of milk flow when the baby is sucking on the nipple-shaped protrusion.

In such a device, the at least one portion of the path may be disposed in a region radially remote from the nipple-shaped protrusion, such that the at least one portion of the path lies outside of a region where the lips of the baby may obscure it. Additionally, the at least one portion of the at least one passageway may be transparent or translucent, and disposed in a region radially remote from the nipple-shaped protrusion, such that the flow of milk is visible through the at least one portion. Furthermore, the at least one portion of the at least one passageway may comprise a material which changes color when exposed to milk and is disposed in a region radially remote from the nipple-shaped protrusion, such that the flow of milk is rendered visible by such a color change. As an alternative implementation, the at least one portion of the at least one passageway may comprise an element which emits a sound when a predetermined flow of milk exists within the at least one portion.

In yet another implementation, any of the above mentioned devices may further comprise a ring formed around a base region of the nipple shaped protrusion, the ring having lower flexibility than the flexible layer of material, such that the device remains more readily latched to the breast when the baby ceases sucking. Furthermore, the above mentioned devices should be electronics-free, such that the risk of electro-magnetic radiation in the region of the baby is reduced.

According to yet another implementation of the devices of this application, there is further provided a device adapted to be fitted over at least the nipple region of a woman's breast, the device comprising:

a flexible layer of material having a nipple shaped protrusion, with an inner surface adapted to face the breast of the woman, and an outer surface adapted to face the mouth of a baby, the flexible layer comprising:
(i) a first at least one passageway leading from the inner surface of the nipple shaped protrusion to a location in an outer region of the flexible layer remote from the nipple shaped protrusion, and a second at least one passageway leading from the location in the outer region of the flexible layer remote from the nipple shaped protrusion, to the outer surface of the nipple shaped protrusion, at a location in the apex region of the nipple shaped protrusion; and
(ii) a fluid connector port at the remote outer region of the flexible layer, having fluid connection openings positioned so that the openings connect to the first at least one passageway, and to the second at least one passageway,
   wherein the fluid connector port is configured for attachment thereto of a head providing information relating to the milk supply transferred though the device from the woman's breast to the baby.

In such a device, the head provides information regarding the quantity of milk flowing from the woman's breast to the baby. The head may comprise a miniature flow sensor, or a container connected by a channel to the path of the milk flow in the head, such that a medication can be added to the milk flow, or a vacuum indicator, such that the sucking level of the baby may be determined, or an analysis sensor, such that a content of the milk may be determined. Additionally, the head may comprise a transmission unit for sending the determined information to a remote device.

According to yet another embodiment of the present disclosure, there is provided a nipple shield for use by a nursing woman, comprising:
(i) a flexible layer adapted to fit over at least a portion of the woman's breast, the flexible layer having an inner surface and an outer surface, and a protrusion region adapted to be positioned over the nipple of the woman's breast;
(ii) at least one opening in the protrusion region of the flexible layer, such that at least one passage is formed connecting the inner surface with the outer surface to enable flow of milk from the woman's breast to a baby sucking on the nipple shield; and
(iii) a valve disposed in the at least one passage, the valve allowing flow of milk through the at least one passage from the woman's breast to the baby, but limiting the inflow of air or milk from the outside to the inner surface.

In such a nipple shield, each valve may have an opening pressure level that limits the inflow of air or milk to a predetermined sub-atmospheric pressure. Additionally, each valve may be adapted to maintain a sub-atmospheric pressure in a space between the inner surface and the woman's breast. That sub-atmospheric pressure may be adapted to enable the nipple shield to remain attached to the woman's breast when the baby stops sucking. Alternatively and additionally, the sub-atmospheric pressure may be adapted to reduce the effort required by the baby to obtain milk, or it may be adapted to induce a greater amount of milk from the woman's breast.

In further implementations, such a nipple shield may further comprise at least one element for indicating flow of milk, each of the at least one element being disposed within a passage, such that the nursing woman may be informed that the baby is being supplied with milk. The nipple shield may further comprise a container fluidly connected to the passage, such that a fluid within the container can be provided to the baby. Any of the above described nipple shields may further comprise a ring formed around the base region of the concave region of the flexible layer, the ring having lower flexibility than the flexible layer of material, such that the nipple shield remains more readily latched to the woman's breast when the baby ceases sucking.

According to yet a further implementation, there is provided a method for assisting a woman to nurse a baby, the method comprising:
(i) fitting a nipple shield device over the woman's breast, the nipple shield having at least one first opening facing the woman's nipple fluidly connected by a passage to at least one second opening facing away from the woman's breast; and
(ii) allowing a baby to suck on the at least one second opening, such that milk flows from the at least one first opening and out of the at least one second opening,
   wherein the nipple shield device comprises at least one valve, each being disposed in one of the at least one passage, allowing flow of milk from the woman's breast to the baby, but limiting the inflow of air or milk from the outside to the space between the nipple shield and the woman's breast.

In this method, each valve has an opening pressure that limits the inflow of air or milk to a predetermined sub-atmospheric pressure. The valve may be adapted to maintain a sub-atmospheric pressure in the space between the nipple shield and the woman's breast. That sub-atmospheric pressure may be adapted to enable the nipple shield to remain attached to the woman's breast when the baby stops sucking. Furthermore, the sub-atmospheric pressure may be adapted to reduce the effort required by the baby to obtain milk, or it may induce a greater amount of milk from the woman's breast. Furthermore, the method may be implemented by using a nipple shield device comprising an element for indicating flow of milk, the element being disposed between in at least one passage, such that the nursing woman may be informed that the baby is being supplied with milk.

According to yet another implementation described in this application, there is provided a device adapted to provide an indication of milk flow from a woman's breast to a feeding baby, the device comprising:

a flexible layer adapted to fit over at least a portion of the woman's breast, the flexible layer comprising:
(i) a dome-shaped part adapted to be positioned over the nipple of the woman's breast, and shaped such that when the device is worn by the woman, the dome-shaped part forms a space between the woman's nipple and the inner surface of the dome-shaped part; and
(ii) at least one fluid communication passageway connecting the space formed within the dome-shaped part with at least one closed vessel disposed in a region of the device which is not expected to be hidden by the mouth of a baby feeding at the outer surface of the dome-shaped part, such that milk moving within the at least one closed vessel provides an indication of a flow of milk from the woman to the baby.

In such a device, the at least one closed vessel may be either transparent or translucent, such that motion of milk in the at least one closed vessel is visible. This visual indication of the presence of milk in the at least one closed vessel may be provided by a material which changes color on contact with milk.

Furthermore, such a device may further comprise a ring formed around the base region of the dome-shaped part, the ring having lower flexibility than the flexible layer, such that the device remains more readily latched to the woman's breast when the baby ceases sucking. Such a device may further comprise a valve disposed in the region between the inner surface and the outer surface of the domed part, the valve adapted to allow flow of milk from the woman's breast to the baby, but limiting the inflow of air or milk from the outside to the inner surface.

Any of the above described devices should be electronics-free. Thus, the electronics-free status of the device reduces the risk of electro-magnetic radiation in the region of the baby.

In yet another implementation described in this disclosure, there is provided a two-way valve, comprising:
(i) a body having an axial channel adapted to allow fluid flow through it,
(ii) a flexible diaphragm having an aperture at its inner axial region, the diaphragm being disposed across the intended flow axis of the valve,
(iii) a first restraining element disposed in a first axial direction from a first side of the diaphragm, and configured to limit the flexing motion of the inner region of the diaphragm in the first direction, and
(iv) a second restraining element disposed in the second axial direction from the second side of the diaphragm, and configured to limit the flexing motion of the peripheral outer region of the diaphragm in the second direction.

Such a two-way valve may further comprise a post disposed in the axial channel of the body, on which post the flexible diaphragm may be mounted, wherein the first restraining element is an enlarged section of the post having an outer diameter larger than that of the post, the flexible diaphragm being free to flex in the first direction only from a point beyond that outer diameter. In this valve, the second restraining element may be a narrowed section of the body having an inner diameter smaller than that of the axial channel of the body, the flexible diaphragm being free to flex in the second direction only from a point within that inner diameter. Furthermore, the distance between the outer diameter of the enlarged section of the post, and the outer periphery of the flexible diaphragm may determine the opening pressure characteristics of the valve for flow in the first axial direction. Additionally, the distance between the inner diameter of the narrowed section of the body, and the inner periphery of the flexible diaphragm may determine the opening pressure characteristics of the valve for flow in the second axial direction.

Another implementation of a two-way valve may comprise:
(i) a body having an axial channel and a stepped surface formed in a recessed first end,
(ii) a post mounted in the axial channel of the valve body, forming an annular flow channel within the axial channel of the valve body, the post having a widened extremity forming a stepped shoulder, the stepped shoulder facing a direction opposite to that of the stepped surface of the recessed first end of the valve body, and at a longitudinal position close to the same axial region as the stepped surface of the recessed first end of the valve body, and
(iii) a flexible diaphragm mounted on the post, such that it is positioned between the stepped shoulder of the post and the stepped surface of the valve body, and across the annular flow channel.

In such a two-way valve, the position of the flexible diaphragm may be such that a flow of fluid along the annular channel towards the recessed first end of the valve body is operative to generate a bending motion of the outer periphery of the flexible diaphragm towards the recessed first end, while a flow of fluid along the annular channel away from the recessed first end of the valve body is operative to generate a bending motion of the inner periphery of the flexible diaphragm away from the recessed first end. In such a two-way valve, for a given fluid flow, the extent of the bending motion of the flexible diaphragm towards the recessed first end may be dependent on the radial distance between the step in the stepped shoulder of the of the central post, and the outer periphery of the flexible diaphragm. Additionally, the extent of the bending motion of the flexible diaphragm away from the recessed first end may be dependent on the radial distance between the step of the stepped surface formed in a recessed first end, and the inner periphery of the flexible diaphragm.

In any of these two-way valves, selection of the length of the free diameter of that part of the diaphragm that can bend enables adjustment of the pressure across the valve at which the valve opens. The opening of the valve may be self-actuated by the pressure difference across the valve.

### BRIEF DESCRIPTION OF THE DRAWINGS

The present invention will be understood and appreciated more fully from the following detailed description, taken in conjunction with the drawings in which:

FIG. 1 illustrates schematically an isometric view of an exemplary device for displaying the milk flow of a mother while breast feeding a baby;

FIG. 2A is a plan view of the device shown in FIG. 1; FIG. 2B is a plan view of a device similar to that of FIG. 1, but using an enclosed cavity as an alternative method of indicating the milk flow; FIG. 2C shows the use of a mechanical protrusion to indicate milk flow by deflecting in the flow; FIG. 2D shows an auxiliary container vessel attached to the milk passageway, which fills partly with milk and then partly empties as the baby sucks and rests;

FIG. 3 shows the addition of a valve in the passageway, to improve the functioning of the devices of FIGS. 1 and 2;

FIG. 4A is a schematic illustration of a nipple shield incorporating a valve according to the present disclosure, while FIGS. 4B and 4C illustrate different examples of the structures of the valves used in the devices having valves installed, and FIGS. 4D and 4E illustrate an additional strengthening feature of the nipple shield in the form of a belt, to enable it to remain latched onto the mother's breast more securely;

FIGS. 5A to 5D show one example of a novel differential two-way valve construction, which could be used, inter alia, in the devices of the present application, and FIG. 5E shows the type of opening characteristic obtainable from such a differential two-way valve;

FIGS. 6A and 6B illustrate schematically a medicine dispenser implementation of the device shown in FIGS. 1 and 2;

FIG. 7 is a schematic external isometric view of one example of the base unit of a multi-task nipple shield of the present disclosure, that can be used for performing a number of alternative functions related to different aspects of a nursing mother's needs;

FIG. 8 is an enlarged view of the outside tip region of the nipple protrusion of the device of FIG. 7;

FIGS. 9A and 9B illustrate a method of facilitating the manufacture of the base unit of the multi-task nipple shield of FIGS. 7 and 8;

FIG. 10 is a montage showing how the base unit of a multi-task nipple shield of the present disclosure is used together with various attachment heads available for various different tasks related to the mother's milk supply;

FIGS. 11A to 11D illustrate in greater detail, an exemplary use of the multi-task nipple shield of FIGS. 7 to 10; and

FIG. 12 schematically shows an external view of a schematic implementation of a milk measurement head connected to the base unit of the multi-task nipple shield of FIGS. 7 to 10.

### DETAILED DESCRIPTION

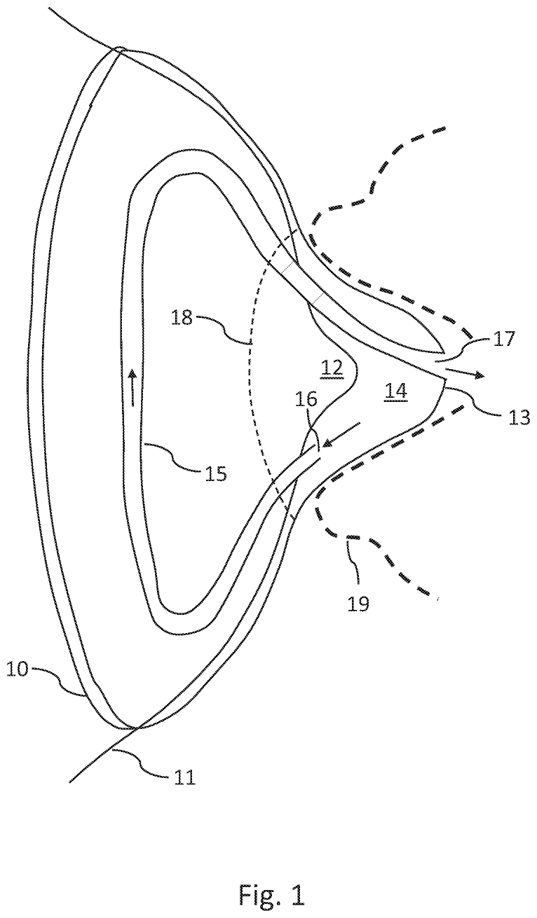

Reference is now made to FIG. 1, which illustrates schematically a side-elevation and partly isometric view of an exemplary device 10 constructed according to the present disclosure, for visually displaying the milk flow of a mother breast feeding a baby. The nipple-shield shaped device 10 has a passageway for indicating milk flow, and is shown fitted over the breast 11 of a woman. During breastfeeding, milk flowing through the passageway is visible to the mother or to a third party such as a nursing adviser, or its presence is indicated by other than a visual method, thus reassuring the mother that the baby is receiving a flow of milk from her breast.

The thin body of the device 10 is typically constructed from a flexible material and has an inner surface which, when worn, faces the breast 11 of the mother, and an outer surface adapted to face away from the mother, such that during breastfeeding, the outer surface faces the lips and mouth 19 of the baby. The flexible material may be a silicone or any other material which is non-absorbent and sufficiently flexible to be worn comfortably by the mother. The flexible material, or at least a part of it, is typically transparent, or semi-transparent, for reasons that will be explained below. Similar to nipple shields currently available, the device 10 when worn covers at least the mother's nipple 12, and typically at least a large portion of the areola, if not the entire areola, as well. The device may cover an even larger portion of the breast than the areola, and generally has a circular, or oval shape, although it may have another shape. The device 10 has a concave region, also appearing as a dome-like protrusion 13 or “the device nipple”, in accordance with its form when viewed from the outside. This region is adapted to be positioned over the nipple region of the mother. The nipple region of the mother is understood to mean the mother's nipple 12, although it may include some of the area surrounding the mother's nipple as well. The device's nipple 13 is typically formed such that when the device is worn, there is a cavity 14 formed between the protrusion 13 of the device and the nipple 12 of the mother. The concave region within the protrusion may be tunnel-shaped, similar to the region known as the “shield tunnel” of commercially available nipple shields. Alternatively, it may be semi-spherically shaped, or may mimic the shape of a mother's nipple 12, as shown in the example of FIG. 1. The outer region of the device surrounding the device nipple 13, is adapted to fit conformally over the woman's breast, such that when the device is worn, the outer region remains close to the woman's breast without an appreciable space therebetween. The approximate boundary region between the device protrusion 13 and the outer regions of the device is shown schematically in FIG. 1 by the faint dashed line 18.

The exemplary implementation of the device shown in FIG. 1 includes a passageway 15 connecting the cavity region 14 with the outer surface at the apex of the protrusion 13, and routed such that at least a portion of the passageway is located in the outer region of the device. The passageway could be formed within the thin flexible material layer of the device, or it could be formed on the surface of the thin flexible material layer of the device, or it could be a separate section of tubing outside of the thin flexible layer and connected to the device between the cavity end and the device nipple end.

During breastfeeding, the device 10 is fitted over the breast 11. The passageway 15 has an entrance aperture 16 in communication with cavity 14, and an exit aperture 17 at the device nipple 13. The outline of the baby's lips 19 sucking on the device nipple, are shown in FIG. 1 as dashed lines. Negative pressure generated by the sucking actions of the baby on the device, or the mother's physiological reaction to the baby's tongue movements, cause milk to be expressed from the breast of the mother into the cavity 14, from where it is drawn through passageway 15 to exit the device through exit aperture 17. All of the milk supplied to the baby thus flows along the passageway 15, as shown by the arrows in FIG. 1. The device is intended to be worn with the entrance of the passageway 16 at the bottom, such that milk collected by gravity at the bottom of the cavity 14 will be efficiently collected. For this reason, the device may be constructed with the cavity 14, being the milk collection volume, concentrated at the bottom region of the space between the mother's nipple and the device nipple over the entrance aperture 16 of the passageway 15.

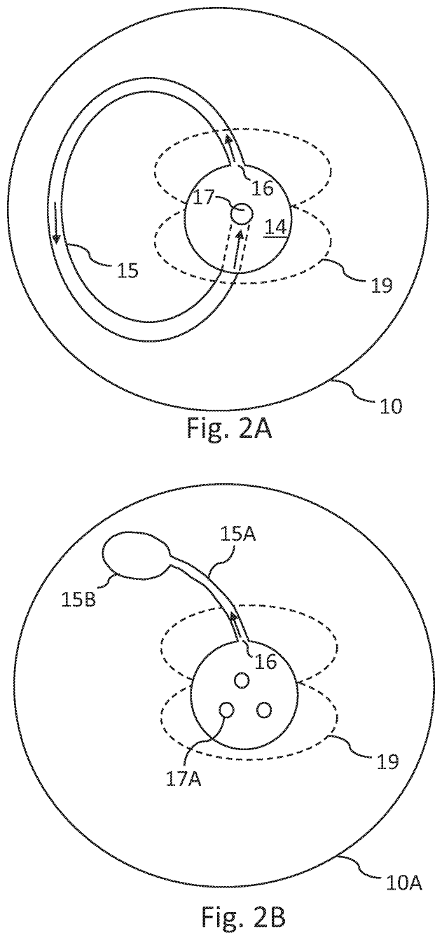

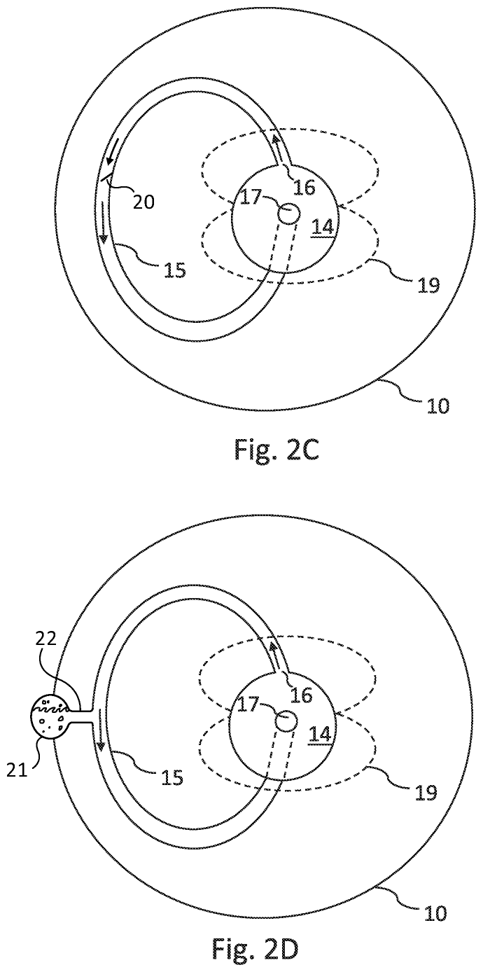

Reference is now made to FIG. 2A, which is a plan view of the device shown in FIG. 1, illustrating more clearly the course of the passageway 15. The numbering of the features in FIG. 2A are identical to those of FIG. 1. The passageway 15 is routed such that it runs from the cavity region 14 in the protrusion, in an outward path into the outer region of the device. By this means, it passes out from the region beneath the lips 19 and mouth of a sucking baby, where it would be hidden from the view of the mother, into a region where its view is not obstructed by the lips or mouth of the baby and where the mother or an assistant can see the flow of the milk in the passageway. The passageway may be directed radially outwards towards the rim of the device, as in the example shown in FIG. 2A. On its return path from the outer region of the device, the passageway re-enters the cavity, while maintaining fluid isolation therefrom, and ends at the exit aperture on the outside apex of the device nipple, where the milk is provided to the baby.

The walls of the passageway are typically transparent or translucent, such that when milk is flowing through the passageway, a person viewing the device receives an indication whether milk is indeed being drawn from the breast and supplied to the baby. In the case wherein the flexible material is not transparent or translucent, at least a portion of the flexible material surrounding the passageway in an area not expected to be obscured by the baby during breastfeeding, should be made of transparent or semi-transparent material. If the passageway is not formed within the flexible material, then a section of the tubing of the passageway itself should be transparent or translucent, to show the flow within the passageway. As an alternative to visual observation of the flow itself through the walls of the passageway, the passageway, according to other exemplary implementations, can be manufactured of an indicator material, such a material providing a color change when contacted by milk. Another implementation could use a feature or protrusion in the passageway, such as a reed, or a flap, or a paddle-wheel, which generates a sound when the milk flows past or through it.

Though the simplest way of forming the passageway is by forming it within the flexible layer, the device can also be constructed using a tube which either passes outside of the flexible layer of the device, or is attached to its surface, usually the outer surface so as not to interfere with the airtightness of the device relative to the mother's breast. The passageway typically has the shape of a tube, such that it has a generally circular cross-section, though other cross sections may be used without affecting the usefulness of the device.

Reference is now made to FIG. 2B, which illustrates an alternative structure of the milk flow detector 10 of FIG. 1, in which the indicator passageway does not have to have the whole, or even a part of the milk flow passing right through it, in order to indicate the presence of a milk flow. In the implementation shown in FIG. 2B, the passageway of the device 10A takes the form of a short spur 15A, leading from its entrance aperture 16 in the cavity 14, to a small enclosed volume 15B, preferably transparent or translucent, where milk may collect. The actions of the baby alternatively sucking and relaxing, causes milk to, at least partially, enter and exit the closed indicator element 15B, or to splash about in it under the influence of the alternating pressure fluctuations acting on the milk in the spur 15A. The mother can then see the milk entering and leaving the indicator chamber 15B and can thus become informed that the baby is getting his or her milk flow. The mother can learn to distinguish between different characteristics of the way in which the milk is splashing about in the indicator chamber 15B, or into and out of it, and to relate that appearance to the rate at which the baby is taking the milk. The actual flow of milk from the mother's nipple to the baby's mouth 19, takes place through nipple openings 17A of the device, as in any conventional nipple shield.

Reference is now made to FIG. 2C, which shows an additional method by which the flow of milk through the passageway can be indicated. In the embodiment of FIG. 2A, there was mentioned above how a feature or protrusion in the passageway, such as a reed, or a flap, or a paddle-wheel, could be used as a sound generating element, emitting a characteristic sound when the milk flows past or through it. However, in addition to the auditory output generated by such an element, a physical visual indication of the presence of milk flow within the passageway could also be provided. Thus for instance, as shown schematically in FIG. 2C, a flap 20 extending into the flow across the passageway 15, is deflected by the flow of the milk onto its upstream surface, and this deflection may be clearly visible from the outside of the passageway.

Reference is now made to FIG. 2D, which illustrates yet another implementation for providing indication of milk flow through the passageway. In the implementation shown schematically in FIG. 2D, the passageway comprises a small enclosed container vessel 21 attached to the passageway 15 by means of a short connecting tube 22. Thus, when the milk flows past the attached container connecting tube 22, it surges into the container vessel 21, providing a visual indication of the flow of the milk in the passageway. The visual indication is even more strongly emphasized, since the baby draws milk in pulses, during every sucking period, and rests between sucking actions. Consequently, during the sucking period, the milk flowing along the passageway surges into the container, possibly only partly filling it, and then when the baby relaxes between his/her suction actions, part of the milk in the container flows back out into the passageway. This constant surge of milk into and out of the container, possibly including bubbles generated in the surge, provides a good visual indication of the flow of the milk as the baby sucks. In FIG. 2D, the container vessel is shown horizontal, but it is to be understood that the device may be used such that the container is situated in a vertical position, so that the milk is clearly seen filling up and emptying from the container vessel

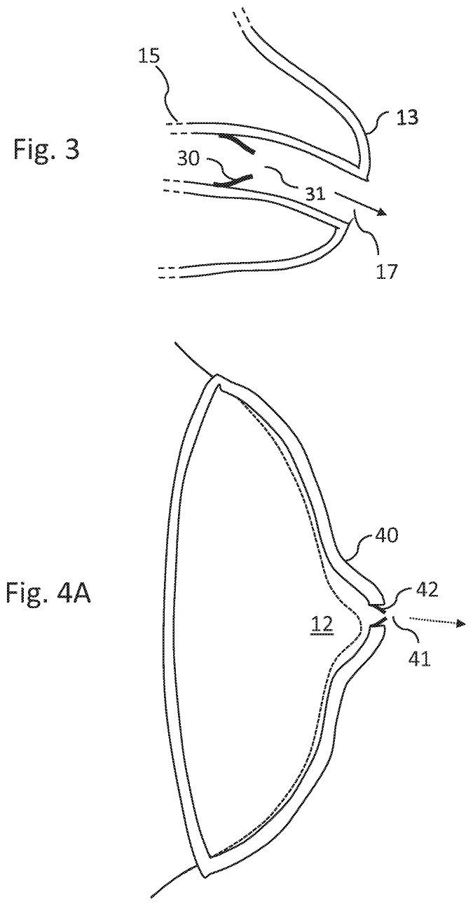

Reference is now made to FIG. 3 which shows a further embodiment in which a valve 30 is fitted into the passageway 15, to assist in the maintenance of a sub-pressure in the passageway and the cavity, so that the device remains attached to the mother's breast, as explained in the Summary section hereinabove. In FIG. 3, showing a typical example of such an implementation, the valve 30 is shown in this example as a one-way valve in the form of a diaphragm valve or a flap valve which closes when suction is not applied by the baby. In FIG. 3, which is a cross-sectional view, the flap-valve 30 is shown as a circular diaphragm with a central aperture 31, which opens when a forward flow of milk passes through it, but which closes when the milk flow stops due to the baby ceasing to suck, and thus maintains the sub-pressure within the passageway 15. It is to be understood however, that any other form of valve may also be used, without affecting the functionality of the device. Similarly, although the valve in FIG. 3 is fitted near the exit 17 of the passageway, at the device nipple 13, it may equally well be formed at any other point in the passageway's path.

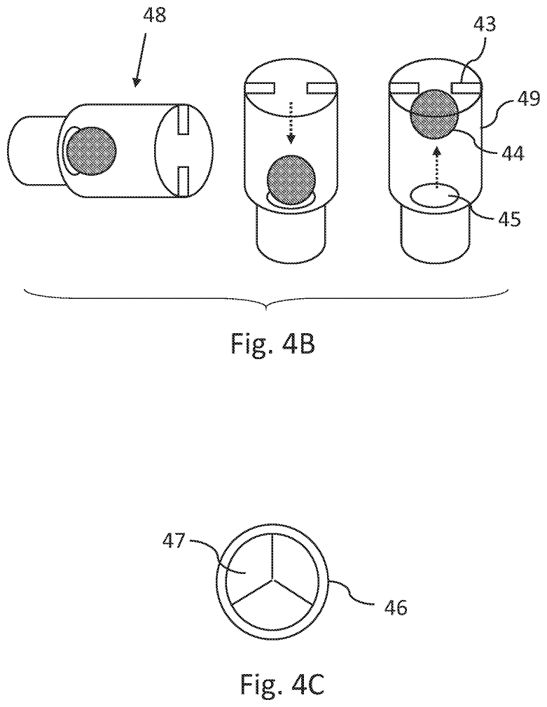

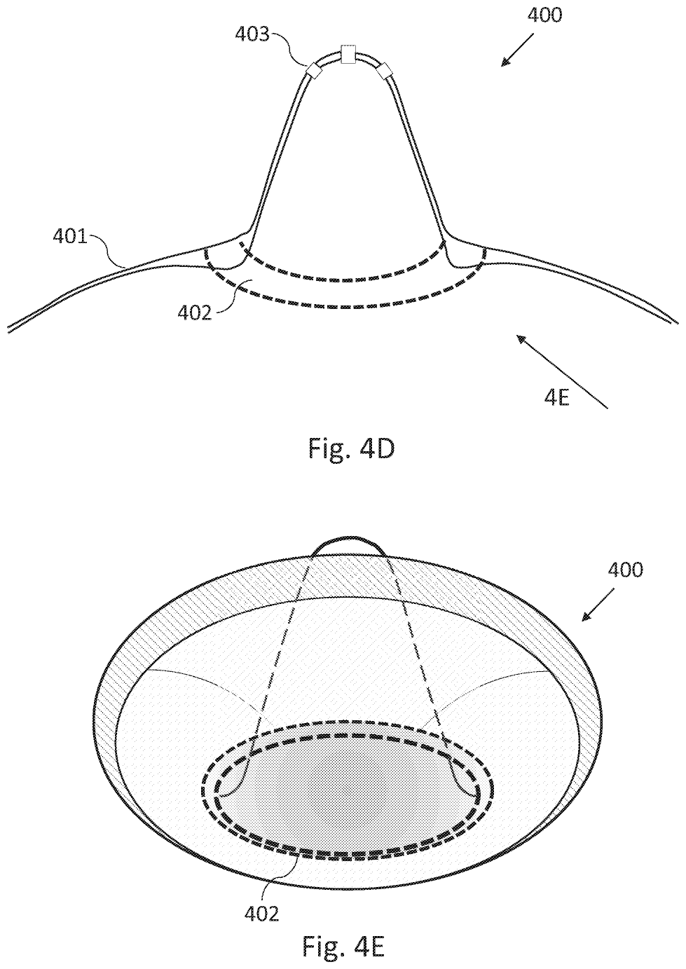

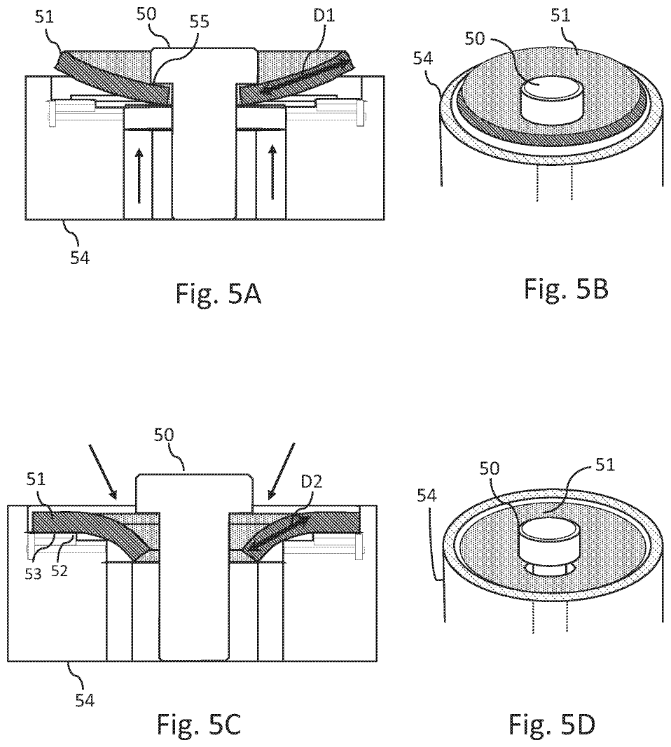

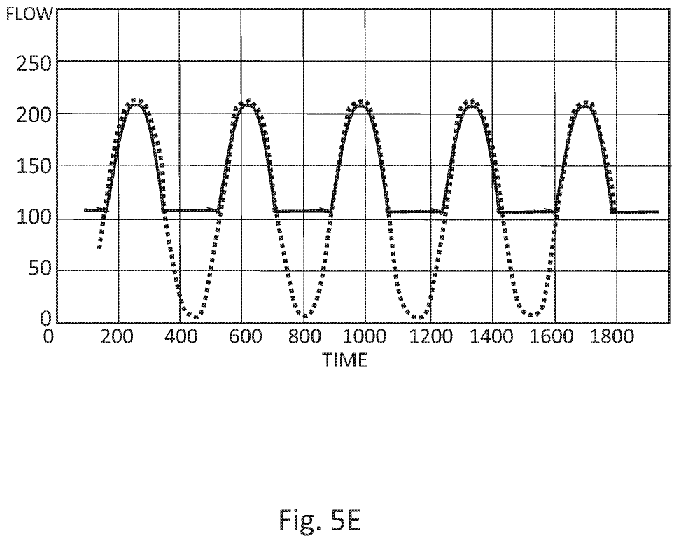

Reference is now made to FIG. 4A, which is a schematic illustration of a novel nipple shield 40, constructed according to another exemplary embodiment of the present application. The nipple shield incorporates a valve 42. This improved nipple shield 40 is shown mounted on the nipple 12 of the breast of the mother using it, and the baby receives the mother's milk through the opening 41, shown in this drawing as a single opening, though there could be more than one opening, each with its own valve. When the baby stops sucking, the valve 42 closes, thereby preventing the inflow of air to any space between the nipple shield 40 and the mother's breast 12, thereby assisting in ensuring that the nipple shield remains affixed to the mother's breast. The valve 42, is shown as a simple diaphragm or flap valve, but could be any other type of valve which will provide the sealing of the nipple passage 41 required. The use of such a valve in this nipple shield, improves the ease of using the nipple shield, by ensuring latching of the nipple shield onto the mother's breast, even when the baby takes a pause from his/her sucking actions. Furthermore, as described in the previous embodiments, and as will be more fully described hereinbelow in connection with FIGS. 5A to 5D, the valve could have a two-level opening pattern, such that in the inflow direction, namely, the flow back in the direction from the baby to the mother's breast, the valve could be adapted to require a higher pressure differential to open than the pressure required to keep the valve open in the reverse, outflow direction. This two-way valve allows some inflow of air before closing, to increase the comfort of the mother.

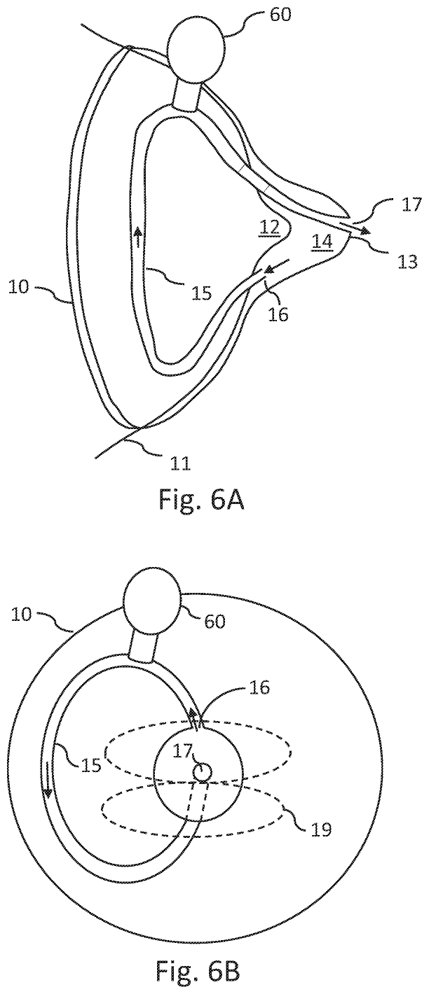

Additionally, the improved nipple shield could also incorporate a fluid dispensing attachment, not shown in FIG. 4A but as described hereinbelow in FIGS. 6A and 6B, and in associated paragraph [0068]. This addition could be used in a simple device to enable the accurate dispensing of a medicine to the baby while the baby is suckling milk.

According to a further implementation of the above-described improved nipple shield of FIG. 4A, the incorporation of a flow indicating element in the short nipple passage 41 of the device, enables the device to provide an indication to the mother that the baby is being provided with a supply of milk, about which she would not otherwise be certain. The flow indicating element could be a sound producing component, since such a component will provide its indication even if hidden in the mouth of the baby.

Reference is now made to FIGS. 4B and 4C which illustrate schematically other exemplary forms of valve which could be used in any of the devices of the present application, whether a milk flow detection device, or a nipple shield incorporating valving, as per the improvement presented in the present application. FIG. 4B shows three different views of a ball valve 48, which may be installed either at the nipple of a valved nipple shield of the present disclosure, or at the exit or somewhere along the length of the milk passageway of the milk flow detection device of the present disclosure. The ball 44 of this valve is able to move along a chamber 49. When the ball 44 sits on an opening 45, which is attached to the internal fluid side of the nipple opening, it closes the aperture 45, and prevents back inflow of air into the device, thereby maintaining its contact with the mother's nipple. When the baby sucks on the nipple, the ball 44 is pushed of the aperture 45 by the flow of milk, thereby enabling the baby to access the flow of milk. The ball has to be prevented from being ingested by the baby, and in the embodiment of FIG. 4B, this is achieved by a pair of tangs 43, which trap the ball within its cavity 49. FIG. 4C shows an alternative valve in the form of a simple diaphragm valve 46, similar to that shown in cross-section in FIG. 4A, in which the flaps 47 open when a flow of milk occurs, but returns to the closed position when the flow of milk ceases. As previously mentioned, any suitable valve can be used for these embodiments.

Reference is now made to FIGS. 4D and 4E, which illustrate a further feature of such a nipple shield 400, in which a band or ring of higher strength is built into the base of the nipple of the nipple shield, such that it is held more securely onto the mother's nipple. In FIG. 4D, there is shown a side cross-sectional view of the nipple region of the device, showing the openings 403 in the nipple dome for the baby to suck on, and the thin flexible outer region 401 of the device, for mounting on the mother's breast. At the junction of the thin flexible outer region and the nipple dome, there is formed a circular band 402 of thicker or less flexible material, which reduces the ability of the device to bend or to lift off from the mother's nipple, thereby assisting in keeping the device attached to the mother's nipple when the baby ceases sucking. Such a strengthening band can also be applied to any of the milk flow indication devices of FIGS. 1 to 4A. FIG. 4E shows a partially isometric view of the whole of the device, viewed from the direction marked 4E in FIG. 4D, to show more clearly the position of the strengthening band 402. Furthermore, any of the devices described in this disclosure, which incorporate a pressure regulating valve, can benefit from such a strengthening band or ring, since maintenance of the vacuum by the valve, when the baby pauses from his/her sucking actions, is enhanced by the use of the above-described band or ring.

Reference is now made to FIGS. 5A to 5D, which show one example of a novel, two-way differential valve 64, having different opening pressure characteristics in the two flow directions. The valve is self-actuated, in the sense that its opening is determined by the differential pressure applied across the valve. Such a valve could be used in the devices of the present application, and its operation is thuswise described in the following paragraphs. It is to be understood however, that such a valve could be used in any other application, whether for liquid or for gaseous flow control, and as such, is not intended to be limited to the application described here within, having wide applications throughout industry and medicine.

FIGS. 5A and 5C show cutaway illustrations of such an exemplary two-way valve, while FIGS. 5B and 5D are isometric views of the two-way valve. As mentioned hereinabove, such a two-way valve is adapted to enable an essentially free flow of the mother's milk to the baby, while at the same time enabling an inflow of air to the mother's nipple when the baby stops suckling, up to a predetermined negative pressure, in order to reduce the level of the negative pressure acting on the mother's nipples. The valve opening pressure in the outward flow direction should occur at a lower operating pressure than the valve closing pressure in the inward flow direction. By this means, the baby can suckle with minimal obstruction once the valve has opened for the outward flow, while the reverse flow for reducing the level of the negative pressure on the mother's nipple, takes place at a higher differential pressure setting, such that the device is still held on the mother's breast.

The valve shown in FIGS. 5A to 5D is a two-way differential valve, self-actuated by the pressure difference across the valve, and the opening pressure may be different for the two directions of flow. The valve uses a flexible diaphragm 51 which is confined in the valve flow passageway, across the direction of the fluid flow, between an outer shouldered stepped structure 53 of the valve body 54, and an inner stepped edge 55 of a pedestal 50 mounted centrally in the valve, where “inner” and “outer” relate to the radial distances from the central axis of the valve. The flexible diaphragm 51 is disposed across the direction of flow of the fluid, and is endowed with a different flexibility for the two directions of flow. This difference of flexibility is generated by providing a different bending length of the diaphragm, between the point of support of the diaphragm in the valve body 54, and its freely movable inner or outer periphery. The diaphragm is constrained between the inner post 50 having an annular shoulder 55 constraining the inner edge of the diaphragm, such that the outer edge of the diaphragm can only bend in one direction, away from the central post annular shoulder 55, and an outer shoulder 52, 53, constraining the outer edge of the diagram, such as the diaphragm can only bend in the other direction, away from the outer shoulder 52, 53. Adjustment of the length of the free diameter of the part of the diaphragm that can bend enables control of the pressure across the valve which causes it to open.

As shown in FIGS. 5A and 5B, when the baby sucks and generates a negative pressure, a flow of milk occurs, as indicated by the upwardly directed arrows in FIG. 5A. The terms “upwards” and “downwards” in this connection, relate to the directions shown in the drawing, and have nothing to do with the absolute direction in space. The outer edge of the flexible diaphragm 51 lifts off from its shouldered stepped structure 53, to enable the milk to flow around its outer edge. Its upward movement is limited by its intrinsic flexibility and by the inner shoulder and stepped corner 55 of the central pedestal 50, around which it bends, and the milk flow takes place around the periphery of the flexed diaphragm. On the other hand, when the baby stops sucking and the flow of milk stops, the negative pressure within the flow passageway, generated by the baby's sucking, causes the flexible diaphragm 51 to be pushed inwards by the external air pressure, from its seating on the stepped structure 53, bending around the stepped corner 52 of that structure. Air, or milk still within the valve passageway or the baby's mouth, then enters the device by flowing between the central pedestal 50 and the internally flexed diaphragm 51, as shown by the downwardly directed arrows in FIG. 5C. The inward flow of air/milk continues until the sub-pressure within the device rises to the level at which the diaphragm 51 closes since the pressure difference across the valve is insufficient to keep it open.

The structure of the two-way valve shown in FIGS. 5A to 5D is such that the pressure required to open the flow of the valve can be different for the two directions of flow. This may be desirable since the difference in pressure needed to open the valve and enable the flow of milk from the mother to the baby, as indicated by the upward arrows in FIG. 5A, should be low, so as not to place an excess burden on the baby's sucking efforts. This pressure difference should generally be less than the pressure difference at which the valve opens in the reverse direction, in which ingress of air or milk is allowed, as shown by the arrows in FIG. 5C, to reduce the level of the vacuum formed within the milk flow passage. This differential opening pressure is achieved by the structure of the valve. which is arranged such that the ease of flexing of the flexible diaphragm 51 is different in the two directions.

As is known, the bending of a flexible diaphragm is dependent on three factors:
(a) The Young's modulus of the diaphragm material;
(b) The second moment of inertia in the direction of the bending; and
(c) The free diameter of the part of the membrane that bends.
   For a specific diaphragm material and shape, factors (a) and (b) are predefined, and therefore, differences in the freedom of bending of the diaphragm is dependent on the free diameter of bending, as will now be explained in paragraph [0066].

Referring now to FIG. 5A, when the milk flow is causing the diaphragm to flex outwards, the free diameter of bending, or the flexing length, D1, extends from the bending point at the corner 55 of the central pedestal 50, to the outer rim of the diaphragm. On the other hand, in FIG. 5C, when the inflow of air or milk is desired, and the diaphragm flexes inwards, the flexing length D2 extends from the bending points at the corner 52 of the stepped shoulder 53 of the valve body, to the inner rim of the diaphragm. Since the bending length D1 for the outward flow direction of FIG. 5A is longer than the bending length D2 for the inward flow direction of FIG. 5C, the resistance to bending is less for the outward flow than for the inward flow, such that the valve opens and closes at a lower pressure difference in the outward flow direction than in the inward flow direction. It is to be understood though, that depending on the specific situation for which the two-way valve is designed, the opening pressures could be arranged to be equal or even reversed, with the specific values determined by the selected flexing lengths, D1 and D2.

Reference is now made to FIG. 5E which is an exemplary graph showing the opening pressure characteristics obtainable from a bidirectional, self-actuated differential valve, as shown in FIGS. 5A to 5D. In the graph, there is shown the flow conduction characteristics of the valve in the two opposite directions, one being shown by the dotted line and the other by the solid line. The abscissa of the graph shows the elapsed time in nominal units, while the ordinate shows a valve flow conduction characteristic, also in nominal units. The pressure is applied to the valve according to a sinusoidal periodic characteristic. In the direction of easier flow, namely the direction in which the valve remains open to a lower differential pressure across the valve, this characteristic being shown by the dotted line, the flow ranges from 0 to 200 units as the pressure rises and falls from its minimum to maximum value. In the direction of more restricted flow, namely the direction in which a higher differential pressure is required to open the valve, this characteristic being shown by the full line, the valve remains shut until the pressure reaches a value when it begins to provide a flow at the 100-unit level, and remains open right up to the 200 unit level.

Reference is now made to FIGS. 6A and 6B, which illustrate schematically an exemplary medicine dispenser implementation for the device of the type shown in FIGS. 1 and 2. FIG. 6A shows a sectional/isometric view of the device, while FIG. 6B shows a plan view. According to this novel feature of these devices, a drug container reservoir 60 is fluidly connected to the milk passageway 15 by means of a connecting tube, such that once the container has been filled with the correct quantity of medication, the flow of the milk will be mixed with the drug in a controlled way, depending on the flow rate of the medicine from the container reservoir to the milk passageway. This rate of flow can be controlled either by a valve, or it can be determined by the fluid resistance of the connecting tube, according to its cross sectional area and the length of the connecting tube, and by the viscosity of the medicament. The timing of the addition of the medicine to the baby's milk can be determined either by a stop-valve in the connecting tube, or by an air inlet valve in the top of the container. The baby will thus receive all of the drug dose, or any other fluid which it is desired to provide to the baby with, mixed with the mother's milk during breast feeding, and at the rate desired, such that even the taste of a drug would be masked. The container reservoir 60 is shown schematically as a balloon shaped volume, but it is to be understood that it could have any suitable form for this purpose, such as a prefilled vial, or a container with a closable lid. The container reservoir may be detachable for easy refilling, or it may be attached during manufacture to the milk flow detection device, as part of a single use disposable device.

Reference is now made to FIG. 7, which is a schematic external isometric view of one example of the base unit of a multi-task nipple shield of the present disclosure, that can be used for performing a number of alternative functions related to different aspects of a nursing mother's needs. The base unit 70 comprises a thin flexible layer, comparable in shape to a conventional nipple shield, and is adapted to be fitted by the mother over her breast. The central region has a protruded nipple volume 71, which is adapted to fit over the mother's nipple. The nipple shield incorporates a pair of passageways 72, 73, embedded in the thin flexible layer of the shield, each of the passageways connecting the tip of the nipple volume to a remote location disposed in a region 75 which will be accessible to the mother or an assistant while the baby is sucking on the nipple of the device. One of these embedded passageways 72 is fluidly connected to the inside surface of the protruded nipple volume 71, and the other embedded passageway 73 is fluidly connected to the outside surface of the protruded nipple volume 71. The first of these passageways 72 is adapted to convey milk from the inside surface of the protruded nipple volume 71 where it is in contact with milk expelled from the mother's nipple, to a location 75 remote from the nipple protrusion, and the other of these passageways 73 is adapted to convey milk from that remote location 75 back to the outside surface of the protruded nipple volume 71 where it can supply that milk to the baby sucking on the outside of the device nipple. At the remote location each of the passageways terminates in a fluid connection pole 76, 77, of a fluid connector port 78, the two poles having known physical dimensions, and being disposed at a known distance apart. Although the simplest and most cost-effective configuration is the use of a pair of passageways, the device could also be constructed using more than a pair of passageways, so long as at least one of the passageways is connected from an outer surface of the nipple protrusion to a fluid connection element at the remote location, and at least another of the passageways is connected from an inner surface of the nipple protrusion to a fluid connection element at the remote location.

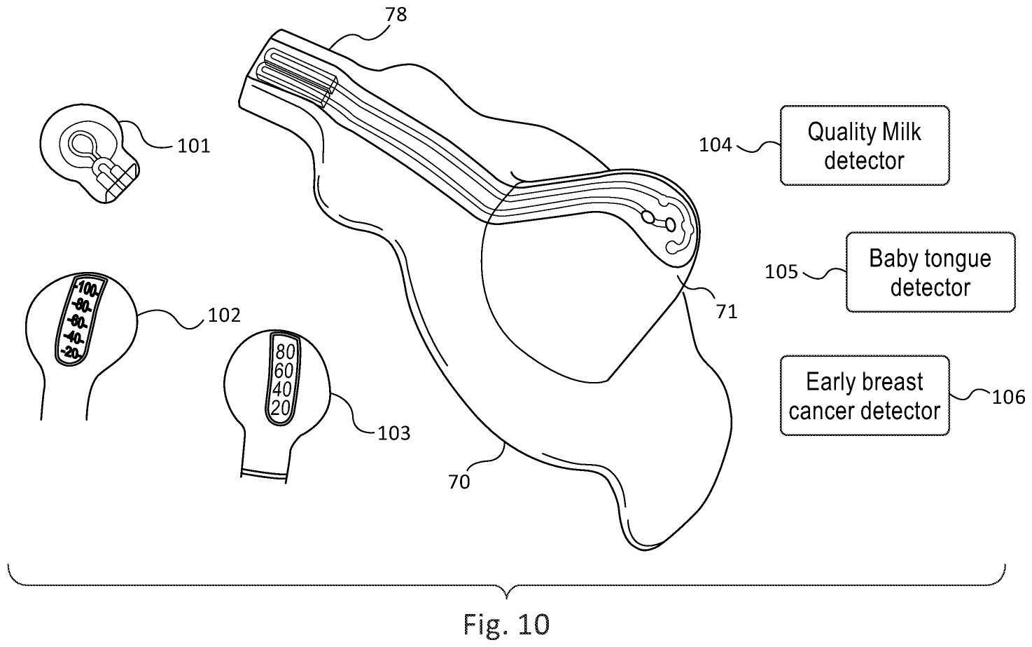

A number of different operational heads, each head being adapted to perform a separate function related to the milk or the milk flow, can be attached to the fluid connector port as will be further shown in FIG. 10 hereinbelow. The base unit 70 of the nipple shield thus has universal uses, and the particular use made of the device depends on the head attached to the remote fluid port 78 of the nipple shield.

Reference is now made to FIG. 8, which is an enlarged view of the outside tip region of the nipple protrusion of the device of FIG. 7, showing how the passageways 72, 73, are connected to the inside surface of the nipple region 71, and the outside surface of the nipple region 71, respectively. As is observed in FIG. 8, the apertures 82 of the first passageway 72 are open to the inside surface, while the apertures 83 of the second passageway 73 are open to the outside surface. Three apertures are shown in each passageway, in order to provide a low resistance to the fluid flow, though it is to be understood that this is simply an exemplary implementation, and that any other number and form of the apertures that provide a suitable flow of milk may be used.

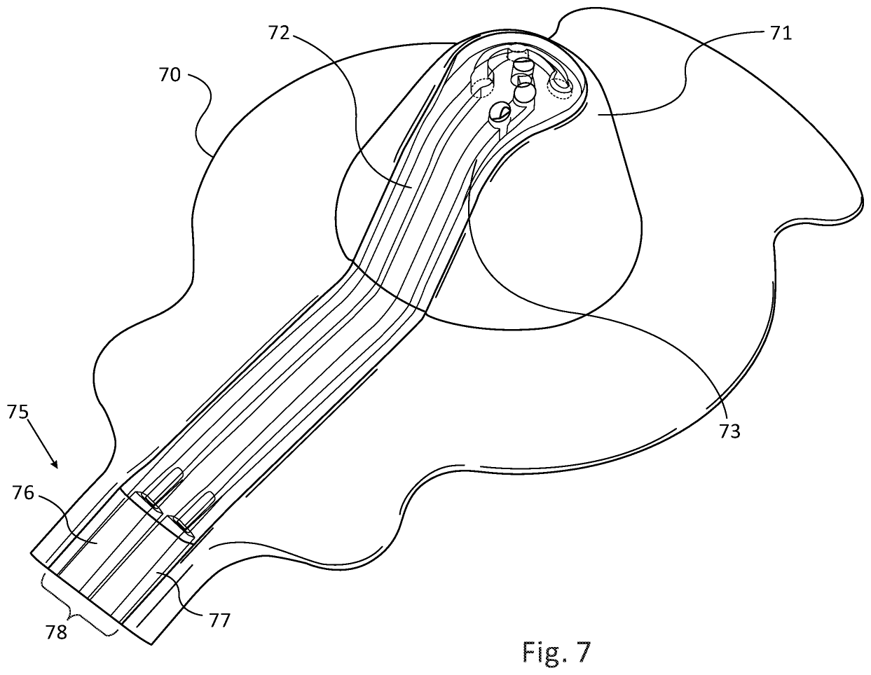

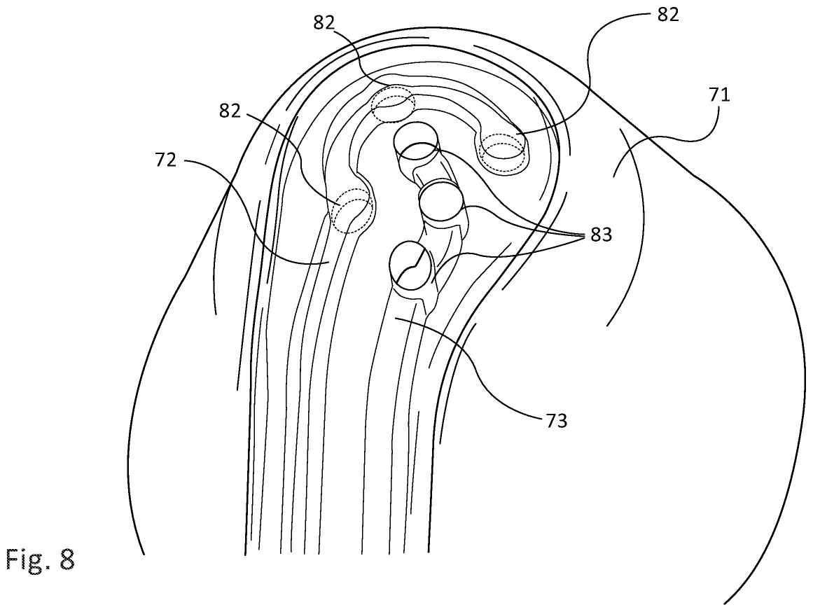

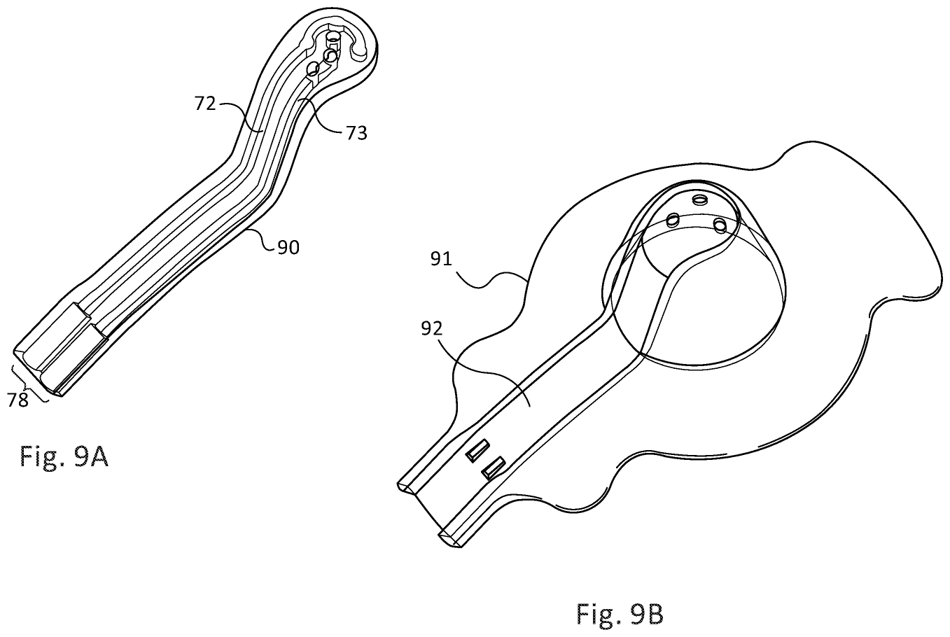

Reference is now made to FIGS. 9A and 9B, which illustrate a method of facilitating the manufacture of the base unit of the multi-task nipple shield of FIGS. 7 and 8. Since the properties required of the section of the device containing the passageways are different from those required of the thin flexible layer of the nipple shield device, it is advantageous to manufacture the passageway section separately from that of the nipple shield itself. FIG. 9A shows a strip section of material 90 containing the molded passageways 72, 73, with the fluid connection port 78, at the remote extremity. FIG. 9B shows the thin flexible body 91 of the nipple shield device, with a shallow channel 92 formed in part of the thickness of the thin flexible body, having a shape adapted to receive the strip section of material 90 containing the molded passageways 72, 73. The tip region of the nipple region of the device has apertures formed therein connecting to the inner surface of the nipple protrusion, the position of the apertures matching the apertures in the first passageway 72 adapted to convey milk from the inside of the nipple volume towards the fluid port 78 at the remote location.

Reference is now made to FIG. 10, which is a montage showing how the base unit 70 of a multi-task nipple shield of the present disclosure is used together with the various attachment heads available for various different tasks related to the mother's milk supply. The attachment heads are adapted to plug into the fluid connection port 78, located remotely from the nipple region 71 of the device. A number of different attachment heads are shown in FIG. 10. All of the heads have one feature in common, namely that connection of the head to the fluid connection port completes the circuit for the milk between passageways 72 and 73, such that the baby can freely suck milk from the mother's nipple, with the milk flowing along the first passageway 72 to the head attached at the fluid port 78, and back through passageway 73 to the baby's mouth. The flow indication head 101, provides an indication to the mother that the baby is receiving a flow of milk through the nipple shield. The flow indication head 101, can use any of the flow indicating features described in the earlier implementations of a flow indicator device of the present disclosure. The flow measurement head 102, is able to make quantitative measurements of the quantity of milk flowing from the mother to the baby, which can be based on the technique described in the previously mentioned U.S. Pat. No. 7,896,835 for “Apparatus and Method for Measuring Fluid Flow to a Suckling Baby”, commonly owned by the present applicant, in which a fraction of the main milk flow is passed through a measurement channel, where the quantity of milk collected in the measurement channel provides a measure of the milk drawn through the main channel. Alternatively, the head 102 can incorporate a micro-technology flow sensor in a loop formed in the head, through which the main stream flows, and the output signal can be transmitted to a remote reader that displays the flow rate, and can integrate the flow rate to provide the quantity of milk delivered. Since the device is intended for domestic use, a transmission system that communicates with, for instance, a smartphone application, would be advantageous.

The head 103 for the addition of medication to the baby's milk feed, can incorporate a small medication enclosure connected by a channel to the milk passageway, such that the medication can be slowly added to the flow of the baby's milk. In addition to the functionalities of the devices previously described in this disclosure, the use of such a universal multitask nipple shield, enables determination of a number of additional measurements and features. Thus for instance, a miniature chemical or spectroscopic analysis head 104, can enable the determination of the quality of the milk or of its various components such as its fat level, or the insecticide content, and similar analyses. A further head 105 may be used for determining the sucking efficiency of the baby, such as by measuring the level of vacuum generated within the head, or the length of a sucking period compared with a rest period of the baby, or other features characterizing the baby's sucking ability. Additionally, an analysis of the mother's milk in a disease detection head 106, which would include a micro-spectrometric or bio-chemical analysis unit, may provide advance warning of an illness or disease, which can manifest itself in the baby's milk delivered from the mother's breast. Such an analysis head may have the potential of early detection of breast cancer of the mother using the device. An advantage of the multitask nipple shield device of FIG. 10, is that all of the above suggested functions can be executed with their relevant head attachments, without interfering in the milk flow provided to the baby.

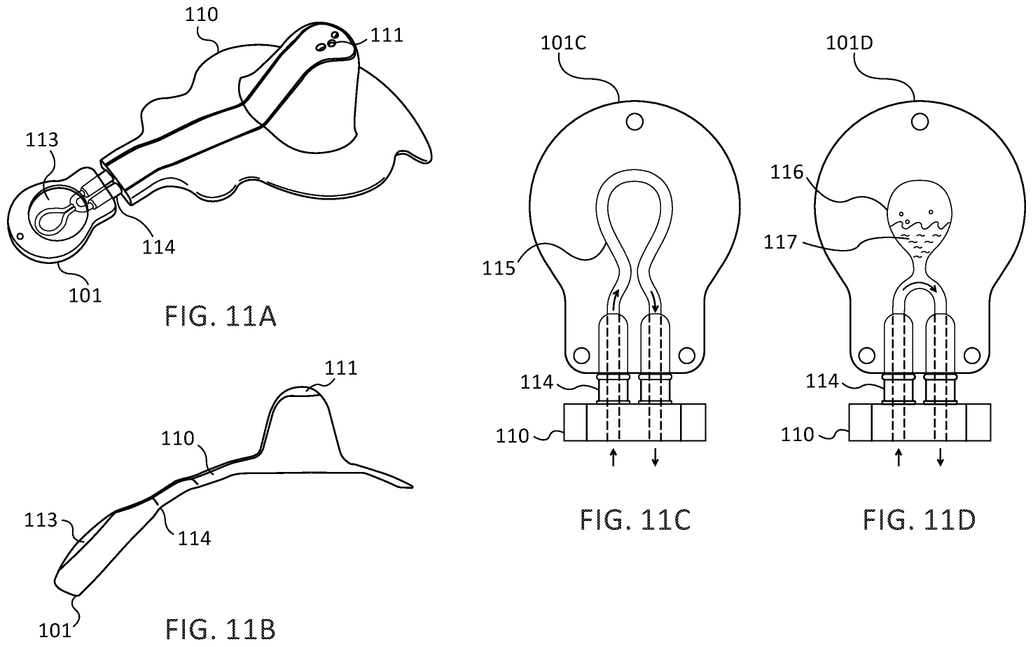

Reference is now made to FIGS. 11A to 11D, which illustrate in greater detail, an exemplary use of the multi-task nipple shield of FIGS. 7 to 10, for the flow indication application using head 101 of FIG. 10. In FIG. 11A, there is shown the base unit 110 of the multi-task nipple shield, with the nipple openings 111 in the apex region of the nipple protrusion, and with the flow indication head unit 101 attached at the fluid port connection 114. The head 101 is shown having a window region 113, which should be transparent in order to facilitate viewing of the flow indication within the attached indicating head 101. In FIG. 11B, there is shown a side view of the base unit 110 with its attachment head 101, to show the way in which the transparent viewing window 113 is positioned remotely from the nipple region 111, so that it can be readily viewed by the nursing mother or an assistant.

Reference is now made to FIGS. 11C and 11D, which schematically show two alternative schematic implementations of the flow indicating head 101, on a larger scale, in order to show the details of the head structure. The two different figures show alternate ways in which the indication of the milk flow can be generated. In FIG. 11C, there is shown a flow indicating head 101C, which uses the method of indicating the flow as shown in FIG. 2A. The flow of milk coming through the passageway in the base unit 110 from the milk collection volume within the nipple protrusion region, is directed around a transparent or translucent loop of tubing 115 within the viewing window 113, and then passes back out of the viewing head into the passageway of the base unit for returning to the nipple openings which on which the baby can suck. The mother can thus have a direct view of the milk flow within the viewing window. In FIG. 11C, there is shown an alternative flow indicating head 101D, which uses the construction shown in FIG. 2D. In this implementation, an indicating milk chamber 116 is fluidly connected to the passageway conveying milk from the base unit and back to the base unit, at a junction within the indicating head unit 101D. As the milk flows through the passageway in the head, part of it enters a closed chamber 116, where it can be viewed through the viewing window. providing a visual indication of the flow of the milk in the passageway. In particular, as the baby repeatedly sucks on the mother's breast, the milk passes down the passageway in spurts, and enters the milk chamber 116 in surges synchronized with the sucking actions of the baby. These constant surges of milk 117 into and out of the milk chamber 116, provide a good visual indication of the flow of the milk as the baby sucks.

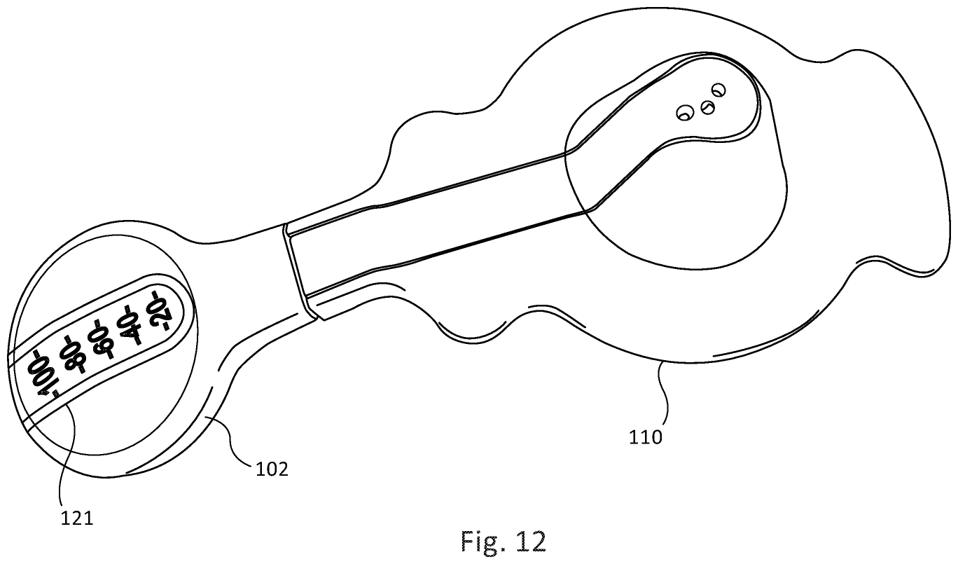

Reference is now made to FIG. 12, which schematically shows an external view of a schematic implementation of the milk measurement head 102, connected to the base unit 110 of the multi-task nipple shield. The head may operate using the same inventive concept as is described in the above referenced U.S. Pat. No. 7,896,835. The structure of such a head unit comprises two flow paths (not shown in FIG. 12) for the milk, the first, which provides the main passageway for milk for the baby, having a substantially lower resistance to the fluid flow of the milk than the second flow path. The second flow path is connected in parallel to the first flow path and has a substantially higher resistance to the milk flow than the first flow path. Consequently, milk flows into the second flow path at a substantially slower rate than in the main path, and the position to which the advancing front surface of the milk reaches, is a measure of the total quantity of milk that has passed through the quantitative measurement head 102. The position of the front of the milk fill can be determined against the graduations 121 on the head, which can be calibrated according to the total quantity of milk taken by the baby. As an alternative implementation, use can be made of a micro-flow meter for measuring the flow rate of milk in the measurement head 102, with a measurement chip for providing an output signal of the flow rate, or by integrating the flow rate, the total quantity of milk consumed by the baby.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. Furthermore, it is appreciated by persons skilled in the art that the present invention is not limited by what has been particularly shown and described hereinabove. Rather the scope of the present invention includes both combinations and sub-combinations of various features described hereinabove as well as variations and modifications thereto which would occur to a person of skill in the art upon reading the above description and which are not in the prior art.

We claim:

1. A device adapted to provide an indication of milk flow from a woman's breast to a baby, the device comprising:
a flexible layer adapted to fit over at least a portion of the woman's breast, the flexible layer comprising:
an inner surface adapted to face the woman's breast, and an outer surface;
a protrusion disposed in a central region of the flexible layer, and adapted to be positioned over the nipple of the breast, and
at least one fluid communication passageway connecting the inner surface of the protrusion with at least one opening in the outer surface in the apex region of the protrusion, such that milk collected in a space between the nipple and the inner surface of the protrusion can flow through the at least one passageway and out of the at least one opening in the apex region, at least one portion of at least one passageway being disposed at a distance from the protrusion, such that during use of the device, the at least one portion is disposed outside of the area where the baby's lips are anticipated to be during use of the device.

2. A device according to claim 1 wherein the at least one passageway is either embedded within the flexible layer, or is partially embedded in the flexible layer, or is disposed on the outer surface of the flexible layer, or is partially detached from the flexible layer.

3. A device according to either of the previous claims wherein at least one part of at least one passageway is either transparent or translucent, such that a flow of milk in the at least one part of at least one passageway is visible.

4. A device according to claim 1 further comprising a closed at least partially transparent container connected to the at least one part of the at least one passageway, such that milk passing through the at least one passageway is visible surging in the at least partially transparent container.

5. A device according to claim 1, wherein the at least one part of the at least one passageway comprises a mechanical element which undergoes deflection in a flow of milk.

6. A device according to any of the previous claims wherein the at least one part of the at least one passageway has a property which indicates when a predetermined flow of milk exists within the at least one part.

7. A device according to claim 6 wherein the property is either a change of color or an emitted sound.

8. A device according to any of the previous claims, wherein the distance of the at least one portion of the at least one passageway from the protrusion, is at least 3 cm., such that the at least one portion is visible outside of the area where the mouth of a baby sucking on the protrusion is expected to be.

9. A device according to any of the previous claims further comprising at least one valve disposed along the at least one passageway and adapted to enable milk to flow through the passageway only from the space to the at least one opening in the apex region.

10. A device according to claim 9, wherein the at least one valve essentially closes when suction is not applied at the least one opening in the apex region.

11. A device according to any of the previous claims, further comprising a container fluidly connected to at least one passageway, such that a fluid in the container can be provided to the baby.

12. A device according to claim 11, wherein the fluid is a medicament.

13. A device according to either of claims 11 and 12, wherein the traversability of the fluid connection is such that the contents of the fluid container are transferred to the milk flow in the at least one passageway according to a predetermined flow rate.

14. A device according to any of the previous claims, wherein the device is electronics-free.

15. A device according to claim 11, wherein the electronics-free status of the device reduces the risk of electro-magnetic radiation in the region of the baby.

16. A device adapted to be fitted over at least the nipple region of a woman's breast, for providing an indication of milk flow from the breast, the device comprising:
a flexible layer of material having a nipple shaped protrusion with an inner surface and an outer surface, the inner surface adapted to face the nipple region of the woman's breast, and the outer surface adapted to face the mouth of a baby; and
at least one passageway formed within the flexible layer, the at least one passageway passing from a first location on the inner surface of the nipple-shaped protrusion, to the outer surface of the flexible layer at or near the apex of the nipple shaped protrusion,
wherein at least one portion of the at least one passageway is adapted to provide an indication of milk flow when the baby is sucking on the nipple-shaped protrusion.

17. A device according to claim 16, wherein the at least one portion of the path is disposed in a region radially remote from the nipple-shaped protrusion, such that the at least one portion of the path lies outside of a region where the lips of the baby may obscure it.

18. A device according to claim 16, wherein the at least one portion of the at least one passageway is transparent or translucent, and is disposed in a region radially remote from the nipple-shaped protrusion, such that the flow of milk is visible through the at least one portion.

19. A device according to claim 16, wherein the at least one portion of the at least one passageway comprises a material which changes color when exposed to milk and is disposed in a region radially remote from the nipple-shaped protrusion, such that the flow of milk is rendered visible by such a color change.

20. A device according to claim 16, wherein the at least one portion of the at least one passageway comprises an element which emits a sound when a predetermined flow of milk exists within the at least one portion.

21. A device according to any of claims 16 to 20, further comprising a ring formed around a base region of the nipple shaped protrusion, the ring having lower flexibility than the flexible layer of material, such that the device remains more readily latched to the breast when the baby ceases sucking.

22. A device according to any of claims 16 to 21, wherein the device is electronics-free.

23. A device adapted to be fitted over at least the nipple region of a woman's breast, the device comprising:
a flexible layer of material having a nipple shaped protrusion, with an inner surface adapted to face the breast of the woman, and an outer surface adapted to face the mouth of a baby, the flexible layer comprising:
a first at least one passageway leading from the inner surface of the nipple shaped protrusion to a location in an outer region of the flexible layer remote from the nipple shaped protrusion, and a second at least one passageway leading from the location in the outer region of the flexible layer remote from the nipple shaped protrusion, to the outer surface of the nipple shaped protrusion, at a location in the apex region of the nipple shaped protrusion; and
a fluid connector port at the remote outer region of the flexible layer, having fluid connection openings positioned so that the openings connect to the first at least one passageway, and to the second at least one passageway,
wherein the fluid connector port is configured for attachment thereto of a head providing information relating to the milk supply transferred though the device from the woman's breast to the baby.

24. A device according to claim 23, wherein the head provides information regarding the quantity of milk flowing from the woman's breast to the baby.

25. A device according to claim 24, wherein the head comprises a miniature flow sensor.

26. A device according to claim 23, wherein the head comprises a container connected by a channel to the path of the milk flow in the head, such that a medication can be added to the milk flow.

27. A device according to claim 23, wherein the head comprises a vacuum indicator, such that the sucking level of the baby may be determined.

28. A device according to claim 23, wherein the head comprises an analysis sensor, such that a content of the milk may be determined.

29. A device according to any of claims 23 to 28, wherein the head comprises a transmission unit for sending the determined information to a remote device.

30. A nipple shield for use by a nursing woman, comprising:
a flexible layer adapted to fit over at least a portion of the woman's breast, the flexible layer having an inner surface and an outer surface, and a protrusion region adapted to be positioned over the nipple of the woman's breast;
at least one opening in the protrusion region of the flexible layer, such that at least one passage is formed connecting the inner surface with the outer surface to enable flow of milk from the woman's breast to a baby sucking on the nipple shield; and
a valve disposed in the at least one passage, the valve allowing flow of milk through the at least one passage from the woman's breast to the baby, but limiting the inflow of air or milk from the outside to the inner surface.

31. A nipple shield according to claim 30, wherein each valve has an opening pressure level that limits the inflow of air or milk to a predetermined sub-atmospheric pressure.

32. A nipple shield according to claim 30, wherein each valve is adapted to maintain a sub-atmospheric pressure in a space between the inner surface and the woman's breast.

33. A nipple shield according to either of claims 31 and 32, wherein the sub-atmospheric pressure is adapted to enable the nipple shield to remain attached to the woman's breast when the baby stops sucking.

34. A nipple shield according to either of claims 31 and 32, wherein the sub-atmospheric pressure is adapted to reduce the effort required by the baby to obtain milk.

35. A nipple shield according to either of claims 31 and 32, wherein the sub-atmospheric pressure is adapted to induce a greater amount of milk from the woman's breast.

36. A nipple shield according to any of claims 30 to 35, further comprising at least one element for indicating flow of milk, each of the at least one element being disposed within a passage, such that the nursing woman may be informed that the baby is being supplied with milk.

37. A nipple shield according to any of claims 30 to 36, further comprising a container fluidly connected to the passage, such that a fluid within the container can be provided to the baby.

38. A nipple shield according to any of claims 30 to 37, further comprising a ring formed around the base region of the concave region of the flexible layer, the ring having lower flexibility than the flexible layer of material, such that the nipple shield remains more readily latched to the woman's breast when the baby ceases sucking.

39. A method for assisting a woman to nurse a baby, the method comprising:
fitting a nipple shield device over the woman's breast, the nipple shield having at least one first opening facing the woman's nipple fluidly connected by a passage to at least one second opening facing away from the woman's breast; and
allowing a baby to suck on the at least one second opening, such that milk flows from the at least one first opening and out of the at least one second opening,
wherein the nipple shield device comprises at least one valve, each being disposed in one of the at least one passage, allowing flow of milk from the woman's breast to the baby, but limiting the inflow of air or milk from the outside to the space between the nipple shield and the woman's breast.

40. A method according to claim 39, wherein each valve has an opening pressure that limits the inflow of air or milk to a predetermined sub-atmospheric pressure.

41. A method according to claim 39, wherein the valve is adapted to maintain a sub-atmospheric pressure in the space between the nipple shield and the woman's breast.

42. A method according to either of claims 40 and 41, wherein the sub-atmospheric pressure is adapted to enable the nipple shield to remain attached to the woman's breast when the baby stops sucking.

43. A method according to either of claims 39 and 40, wherein the sub-atmospheric pressure is adapted to reduce the effort required by the baby to obtain milk.

44. A method according to either of claims 40 and 41, wherein the sub-atmospheric pressure is adapted to induce a greater amount of milk from the woman's breast.

45. A method according to any of claims 39 to 44, wherein the nipple shield device further comprises an element for indicating flow of milk, the element being disposed between in at least one passage, such that the nursing woman may be informed that the baby is being supplied with milk.

46. A device adapted to provide an indication of milk flow from a woman's breast to a feeding baby, the device comprising:
a flexible layer adapted to fit over at least a portion of the woman's breast, the flexible layer comprising:
a dome-shaped part adapted to be positioned over the nipple of the woman's breast, and shaped such that when the device is worn by the woman, the dome-shaped part forms a space between the woman's nipple and the inner surface of the dome-shaped part; and
at least one fluid communication passageway connecting the space formed within the dome-shaped part with at least one closed vessel disposed in a region of the device which is not expected to be hidden by the mouth of a baby feeding at the outer surface of the dome-shaped part, such that milk moving within the at least one closed vessel provides an indication of a flow of milk from the woman to the baby.

47. A device according to claim 46, wherein the at least one closed vessel is either transparent or translucent, such that motion of milk in the at least one closed vessel is visible.

48. A device according to claim 47 wherein the visual indication of the presence of milk in the at least one closed vessel is provided by a material which changes color on contact with milk.

49. A device according to any of claims 46 to 48, further comprising a ring formed around the base region of the dome-shaped part, the ring having lower flexibility than the flexible layer, such that the device remains more readily latched to the woman's breast when the baby ceases sucking.

50. A device according to any of claims 46 to 49 further comprising a valve disposed in the region between the inner surface and the outer surface of the domed part, the valve adapted to allow flow of milk from the woman's breast to the baby, but limiting the inflow of air or milk from the outside to the inner surface.

51. A device according to any of claims 46 to 50, wherein the device is electronics-free.

52. A device according to claim 51, wherein the electronics-free status of the device reduces the risk of electro-magnetic radiation in the region of the baby.

53. A two-way valve, comprising:
a body having an axial channel adapted to allow fluid flow through it;
a flexible diaphragm having an aperture at its inner axial region, the diaphragm being disposed across the intended flow axis of the valve;
a first restraining element disposed in a first axial direction from a first side of the diaphragm, and configured to limit the flexing motion of the inner region of the diaphragm in the first direction, and
a second restraining element disposed in the second axial direction from the second side of the diaphragm, and configured to limit the flexing motion of the peripheral outer region of the diaphragm in the second direction.

54. The two-way valve of claim 53, further comprising a post disposed in the axial channel of the body, on which post the flexible diaphragm is mounted, wherein the first restraining element is an enlarged section of the post having an outer diameter larger than that of the post, the flexible diaphragm being free to flex in the first direction only from a point beyond that outer diameter.

55. The two-way valve of either of claims 53 and 54, wherein the second restraining element is a narrowed section of the body having an inner diameter smaller than that of the axial channel of the body, the flexible diaphragm being free to flex in the second direction only from a point within that inner diameter.

56. The two-way valve of claim 54, wherein the distance between the outer diameter of the enlarged section of the post, and the outer periphery of the flexible diaphragm determines the opening pressure characteristics of the valve for flow in the first axial direction.

57. The two-way valve of claim 55, wherein the distance between the inner diameter of the narrowed section of the body, and the inner periphery of the flexible diaphragm determines the opening pressure characteristics of the valve for flow in the second axial direction.

58. A two-way valve, comprising:
a body having an axial channel and a stepped surface formed in a recessed first end;
a post mounted in the axial channel of the valve body, forming an annular flow channel within the axial channel of the valve body, the post having a widened extremity forming a stepped shoulder, the stepped shoulder facing a direction opposite to that of the stepped surface of the recessed first end of the valve body, and at a longitudinal position close to the same axial region as the stepped surface of the recessed first end of the valve body; and
a flexible diaphragm mounted on the post, such that it is positioned between the stepped shoulder of the post and the stepped surface of the valve body, and across the annular flow channel.

59. The two-way valve of claim 58, wherein the position of the flexible diaphragm is such that a flow of fluid along the annular channel towards the recessed first end of the valve body is operative to generate a bending motion of the outer periphery of the flexible diaphragm towards the recessed first end, while a flow of fluid along the annular channel away from the recessed first end of the valve body is operative to generate a bending motion of the inner periphery of the flexible diaphragm away from the recessed first end.

60. The two-way valve of claim 59, wherein, for a given fluid flow, the extent of the bending motion of the flexible diaphragm towards the recessed first end is dependent on the radial distance between the step in the stepped shoulder of the of the central post, and the outer periphery of the flexible diaphragm.

61. The two-way valve of claim 59, wherein, for the given fluid flow, the extent of the bending motion of the flexible diaphragm away from the recessed first end is dependent on the radial distance between the step of the stepped surface formed in a recessed first end, and the inner periphery of the flexible diaphragm.

62. The two-way valve of any of claims 59 to 61, wherein selection of the length of the free diameter of that part of the diaphragm that can bend enables adjustment of the pressure across the valve at which the valve opens.

63. The two-way valve of any of claims 59 to 62, wherein the opening of the valve is self-actuated by the pressure difference across the valve.
